SEQUENCE LISTING (3 sequences)

SEQ ID NO: 1 - RNA, 21 nt, Artificial
  Synthetic ribonucleic acid sequence
aaccugcugaagaccacagau

SEQ ID NO: 2 - DNA, 21 bp, Artificial
  Synthetic nucleic acid sequence
aataagctccaagagaaaggc

SEQ ID NO: 3 - DNA, 21 bp, Artificial
  Synthetic nucleic acid sequence
aactgccagatggatggtgac

(19) United States
(12) Patent Application Publication
(10) Pub. No.: US 2007/0269406 A1
(43) Pub. Date: Nov. 22, 2007

(54) Transcatheter tumor immunoembolization

(76) Inventor: Thomas E. Ichim, San Diego, CA (US)

Correspondence Address: BAUMGARTNER PATENT LAW, 4370 NE HALSEY ST, STE #124, PORTLAND, OR, 97213, US

(21) Appl. No.: 11/639,395

(22) Filed: Dec. 14, 2006

Related U.S. Application Data
(60) Provisional application No. 60/750,463, filed on Dec. 14, 2005.

(51) Int. Cl. A61K 38/21; A61K 39/00; A61K 45/00; A61P 43/00

(52) U.S. Cl. 424/85.2; 424/184.1; 424/85.5; 424/85.6; 424/85.7

(57) ABSTRACT

Methods of inducing a cancer-specific immune response are disclosed through administration of an immune stimulant in the context of tumor cell death induction. Currently used clinical methods of inducing localized tumor cell death are modified to optimize immune response induction. One embodiment of the invention discloses pharmaceutical compositions and kits for modifying the palliative procedure of transarterial chemoembolization so as to promote uptake and presentation of tumor antigens in an immunostimulatory microenvironment, thereby allowing for induction of T cell, B cell and NK responses, which control not only local, but also systemic tumor growth and metastasis.

### CROSS-REFERENCE TO RELATED APPLICATION

This application claims priority to U.S. provisional application Ser. No. 60/750,463 filed Dec. 14, 2005, the contents of which is incorporated herein by reference in its entirety.

### FIELD OF THE INVENTION

The present invention relates in general to the field of cancer immunotherapy. Specifically, the invention relates to the field of localized immune stimulation for cancer immunotherapy. Even more specifically, the invention relates to the field of initiating, augmenting and maintaining immune responses to antigens released during induced tumor tissue damage.

### BACKGROUND OF THE INVENTION

The focus of cancer research in general is the development of therapies that not only destroy, inhibit, or block progression of primary tumors, but also suppress micrometastatic and metastatic progeny of the primary tumor from seeding the patient. Despite extensive research into the disease, effective means of treating the majoring of cancers at present are elusive to the medical community. Although limited success is achieved using the current standard therapies: chemotherapy, radiation therapy, and surgery; each therapy has inherent limitations. Chemotherapy and radiation therapy cause extensive damage to normal, healthy tissue such as bone marrow, intestinal cells, and even neuronal cells, despite efforts to target such therapy to abnormal tissue (e.g., tumors). Surgery is in many cases effective in removing masses of cancerous cells; however, it cannot always ensure complete removal of affected tissue nor are all tumors in an anatomical location amenable to surgical removal. Furthermore, subsequent to surgical removal, the problem of metastasis and reoccurrence remains unresolved.

Immunological control of neoplasia is suggested by: A) Evidence of longer survival of patients with a variety of cancers who possess a high population of tumor infiltrating lymphocytes (Ryschich, et al. 2005. Clin Cancer Res 11:498-504; Raspollini, et al. 2005. Ann Oncol 16:590-596; Chiba, et al. 2004. Br J Cancer 91:1711-1717, each of which is incorporated by reference herein in its entirety); B) The fact that immune suppressed patients develop cancer at a much higher frequency in comparison to non-immune suppressed individuals (Astigiano, et al. 2005. Neoplasia 7:390-396; Whiteside, T. L. 2004. Cancer Immunol Immunother 53:865-878, each of which is incorporated by reference herein in its entirety); and C) In some very particular situations immunotherapy of cancer is clinically effective (Rosenberg, et al. 2004. Proc Natl Acad Sci USA 101 Suppl 2:14639-14645, which is incorporated by reference herein in its entirety).

While cancer immunotherapy offers the possibility of inducing remission and control of both the primary tumor mass, as well as micrometastasis, several drawbacks exist. The most significant one is that in many situations immunotherapy is either not feasible, or associated with a variety of toxicities. Various types of immunotherapies for cancer have been attempted, including: a) systemic cytokine administration; b) gene therapy; c) allogeneic vaccines; d) autologous vaccine; e) heat shock protein vaccines; f) dendritic cell vaccines; g) tumor infiltrating lymphocytes; h) administration of T cells in a lymphodepleted environment; and i) nutritional interventions. Although each of the approaches contains significant advantages and drawbacks, none of them simultaneously meet the criteria of reproducible efficacy, availability to the mass population, or tumor selectivity/specificity.

The limitations of many immunotherapeutic approaches to cancer is that tumor antigens are either not clearly defined, or in situations where they are defined, the tumor either mutates to lose expression of such antigens, or the antigen-specific vaccine is only applicable to patients with a certain major histocompatibility complex haplotype. The circumvention of this problem has been attempted using autologous vaccines, however in many cases this is an expensive and difficult procedure.

Transarterial chemoembolization (TACE), or otherwise defined as transcatheter chemoembolization, is a clinical procedure used primarily for treating primary and secondary liver cancer (Ramsey, et al. 2002. J Vasc Interv Radiol 13: S211-221, which is incorporated by reference herein in its entirety). TACE is usually employed when standard therapy has failed or is known to be ineffective. TACE combines the advantages of intra-arterial chemotherapy, with the fact that embolization of the portal artery induces a preferential “starvation” of the tumor while sparing non-malignant hepatic tissue. Specifically, it is established that intra-arterial delivery of chemotherapy to the liver results in a tenfold higher intratumoral concentration as compared to administration through the portal vein (Sigurdson, et al. 1987. J Clin Oncol 5:1836-1840, which is incorporated by reference herein in its entirety). This is due in part to the observation that both primary and secondary liver tumors derive their blood supply preferentially from the hepatic artery (Breedis, et al. 1954. Am J Pathol 30:969-977, which is incorporated by reference herein in its entirety). Anecdotal evidence suggested that embolization caused by thrombosis of the catheter during delivery of intraarterial chemotherapy as beneficial for inducing an improved tumor response. This prompted investigators to use surgical ablation (McDermott, et al. 1978. Ann Surg 187:38-46, which is incorporated by reference herein in its entirety) or angiographic embolization (Clouse, et al. 1983. Gastroenterology 85:1183-1186; Nakao, et al. 1986. Radiology 161:303-307; Hwang, et al. 1987. Arch Surg 122:756-759, each of which is incorporated by reference herein in its entirety) to induce localized necrosis. Unfortunately, this approach, in absence of chemotherapy caused little effect on long-term survival. Therefore the advantages of TACE is that both localized delivery of chemotherapy to the tumor occurs, while at the same time, the tumor blood flow is embolized, causing local tumor necrosis (Stuart, K. 2003. Oncologist 8:425-437, which is incorporated by reference herein in its entirety).

Cell death in general is known to release a variety of antigens. Globally speaking, apoptotic cell death is associated with anti-inflammatory and in some cases tolerogenesis, whereas necrotic cell death is perceived by the immune system as a “danger signal”, and is associated with immune activation (Pulendran, et al. 2004. Curr Biol 14:R30-32; Rock, et al. 2005. Springer Semin Immunopathol 26:231-246; McBride, et al. 2004. Radiat Res 162:1-19; Friedman, E. J. 2002. Curr Pharm Des 8:1765-1780; Sauter, et al. 2000. J Exp Med 191:423-434, each of which is incorporated by reference herein in its entirety). Specific examples of the anti-inflammatory aspects of apoptotic cell death include: the production of IL-10 by apoptotic monocytes (Bzowska, et al. 2002. Eur J Immunol 32:2011-2020, which is incorporated by reference herein in its entirety); suppression of inflammatory cytokines by apoptotic bodies in vitro (Cvetanovic, et al. 2004. J Immunol 172:880-889; Hoffmann, et al. 2005. J Immunol 174:1393-1404, each of which is incorporated by reference herein in its entirety), observations that administration of apoptotic but not necrotic cell bodies can actually endow macrophages with active immune suppressive properties (Reiter, et al. 1999. J Immunol 163:1730-1732, which is incorporated by reference herein in its entirety); and clinically administered apoptotic blood cells have been demonstrated successful for treatment of inflammation associated with advanced heart failure in a recent Phase II trial (Torre-Amione, et al. 2004. J Am Coll Cardiol 44:1181-1186, which is incorporated by reference herein in its entirety). Conversely, cellular necrosis is associated with release of a variety of innate immune activation signals such as heat shock proteins (Basu, et al. 2000. Int Immunol 12:1539-1546; Quintana, et al. 2005. J Immunol 175:2777-2782; Tsan, et al. 2004. J Leukoc Biol 76:514-519, each of which is incorporated by reference herein in its entirety), HMGB1 (Rovere-Querini, et al. 2004. EMBO Rep 5:825-830, which is incorporated by reference in its entirety), mRNA with endogenous secondary structures (Kariko, et al. 2004. J Biol Chem 279:12542-12550, which is incorporated by reference in its entirety), and even DNA complexed with endogenous factors such as natural antibodies (Barrat, et al. 2005. J Exp Med 202:1131-1139; Christensen, et al. 2005. J Exp Med 202:321-331, each of which is incorporated by reference herein in its entirety). Therefore the induction of cellular necrosis caused by TACE induces a release of tumor antigens, which is picked up by the immune system. The release of tumor antigens in such situations is reported in the literature (Wu, et al. 2004. Ultrasound Med Biol 30:1217-1222, which is incorporated by reference in its entirety), however taking advantage of this antigen release in the therapeutic context has not been accomplished to date.

Although the in the case of hepatocellular carcinoma, tumor itself (Ormandy, et al. 2005. Cancer Res 65:2457-2464; Jessup, et al. 2004. Clin Exp Metastasis 21:709-717; Yuan, et al. 1997. Cancer Immunol Immunother 45:71-76; Sondak, et al. 1991. Arch Surg 126:442-446), each of which is incorporated by reference herein in its entirety) and host cells infiltrating the tumor are known to be immune suppressive (Griffini, et al. 1996. Clin Exp Metastasis 14:367-380, which is incorporated by reference in its entirety), the microenvironment in which TACE induces cellular necrosis is also normally immune suppressive. It is known that intrahepatic administration of antigens results in systemic immune deviation towards weak cellular immunity (Crispe, et al. 2000. Immunol Rev 174:47-62, which is incorporated by reference in its entirety). For example, it was demonstrated that administration of donor cells into the hepatic circulation resulted in prolonged, donor specific, graft acceptance in various models of transplantation (Kara, et al. 2004. Indian J Med Res 119:110-114; Yu, et al. 1994. Surgery 116:229-234; discussion 234-225; Hamashima, et al. 1989. Nippon Geka Gakkai Zasshi 90:1752-1757; Nakano, et al. 1992. Surgery 111:668-676; Carr, et al. 1996. Ann NY Acad Sci 778:368-370, each of which is incorporated by reference herein in its entirety). The localized immune suppressive effects of the liver are known to the transplant clinician in that liver transplant recipients require a lower degree of immune suppression as compared to other organs. Additionally, in various rodent strain combinations hepatic grafts are spontaneously accepted, while cardiac or renal are rejected (Asakura, et al. 2005. Surgery 138:329-334; Reding, et al. 2004. Liver Transpl 10:1081-1086; Delriviere, et al. 1997. Transplantation 63:1698-1701, each of which is incorporated by reference herein in its entirety). At a cellular level this is explained by the presence of immature hepatic DC (den Dulk, et al. 2003. Arch Immunol Ther Exp (Warsz) 51:29-44; Steptoe, et al. 1997. J Immunol 159:5483-5491, each of which is incorporated by reference herein in its entirety), the tolerogenic potential of liver sinusoidal endothelial cells (Limmer, et al. 2005. Eur J Immunol 35:2970-2981; Onoe, et al. 2005. J Immunol 175:139-146, each of which is incorporated by reference herein in its entirety), as well as natural killer T cells with a predisposition for releasing IL-4 (Crispe, I. N. 2003. Nat Rev Immunol 3:51-62; Sharif, et al. 2001. Nat Med 7:1057-1062, each of which is incorporated by reference herein in its entirety). Based on this, a release of tumor antigens within the hepatic microenvironment is postulated to cause a Th2, or immune regulatory shift, thereby not only failing to initiate protective immunity towards micrometastasis, but in some cases maybe even increasing the rate of tumor growth, through the phenomena of “tumor enhancement” described by Prehn (Prehn, R. T. 1972. Proc Natl Cancer Conf 7:401-404, which is incorporated by reference herein in its entirety).

Accordingly, there exists a need to “reprogram” the local immune environment in areas of tumor antigen release, so as to stimulate a productive immunity, which will cause systemic immunological control of neoplasia.

Although immunological reprogramming of the microenvironment could be accomplished by addition of immune stimulatory factors, the inverse approach of subtracting immunoinhibitory factors is also a possibility. Part of the invention disclosed teaches the use of exploiting the phenomena of RNA interference for localized immune modulation in the context of tumor cell death.

RNA interference (RNAi) is a process by which a double-stranded RNA (dsRNA) selectively inactivates homologous mRNA transcripts. The initial suggestion that dsRNA may possess such a gene silencing effect came from work in Petunias in which overexpression of the gene responsible for purple pigmentation actually caused the flower to lose their endogenous color (Jorgensen, et al. 1996. Plant Mol Biol 31:957-973, which is incorporated by reference herein in its entirety). This phenomenon was termed co-suppression since both the inserted gene transcript and the endogenous transcript were suppressed. In 1998, Fire et al injected C. elegans with RNA in sense, antisense and the combination of both in order to suppress expression of several functional genes. Surprisingly, injection of the combined sense and antisense RNA led to more potent suppression of gene expression than sense or antisense used individually. Inhibition of gene expression was so potent that approximately 1-3 molecules of duplexed RNA per cell were effective at knocking down gene expression. Interestingly, suppression of gene expression would migrate from cell to cell and would even be passed from one generation of cells to another. This seminal paper was the first to describe RNAi (Fire, et al. 1998. Nature 391:806-811, which is incorporated by reference herein in its entirety). One problem present at the initial description of RNAi, and subsequent papers following, was that in order to induce RNAi, long pieces 200-800 base pairs, of dsRNA had to be used. This is impractical for therapeutic uses due to the sensitivity of long RNA to cleavage by RNAses found in the plasma and intracellularly. In addition, long pieces of dsRNA induce a panic response in eukaryotic cells, part of which includes nonspecific inhibition of gene transcription but production of interferon-α (Proud, C. G. 1995. Trends Biochem Sci 20:241-246, which is incorporated by reference herein in its entirety). In 2001, it was demonstrated that after a long dsRNA duplex enters the cytoplasm, a ribonuclease III type enzymatic activity cleaves the duplex into smaller, 21-23 base-pairs which are active in blocking endogenous gene expression. These small pieces of RNA, termed small interfering RNA (siRNA) are capable of blocking gene expression in mammalian cells without triggering the nonspecific panic response (Elbashir, et al. 2001. Nature 411:494-498, which is incorporated by reference herein in its entirety). Several studies published this year have used exogenously synthesized siRNA to block expression of disease associated genes in vitro. Novina et al demonstrated inhibition of HIV entry and replication using siRNA specific for CD4 and gag, respectively (Novina, et al. 2002. Nat Med 8:681-686, which is incorporated by reference herein in its entirety). Suppression of human papilloma virus gene expression in tissue biopsies from women with cervical carcinoma was reported using siRNA specific for the E6 and E7 genes (Jiang, et al. 2002. Oncogene 21:6041-6048, which is incorporated by reference herein in its entirety). The first report of siRNA used in mammalian models is from McCaffrey et al who suppressed expression of luciferase in mice by administration of siRNA using a hydrodynamic transfection method (McCaffrey, et al. 2002. Nature 418:38-39, which is incorporated by reference herein in its entirety). A subsequent study using HeLa cells xenografted on nude mice compared efficacy of gene suppression between AO and siRNA. Consistent with in vitro evidence, in vivo siRNA administration resulted in a more potent and longer lasting suppression of gene expression than obtained with AO (Bertrand, et al. 2002. Biochem Biophys Res Commun 296:1000, which is incorporated by reference herein in its entirety). Silencing gene expression through siRNA is superior to conventional gene or antibody blocking approaches due to the following: 1) Blocking efficacy is potent (Bertrand, et al. 2002. Biochem Biophys Res Commun 296: 1000, which is incorporated by reference herein in its entirety); 2) Targeting gene expression is specific to 1 nucleotide mismatch (Celotto, et al. 2002. Rna 8:718-724, which is incorporated by reference herein in its entirety); 3) Inhibitory effects can be passed for multiple generations to daughter cells (Grishok, et al. 2000. Science 287:2494-2497, which is incorporated by reference herein in its entirety); 4) In vitro transfection efficacy is higher and can be expressed in a stable manner (Brummelkamp, et al. 2002. Science 296:550-553, which is incorporated by reference herein in its entirety); 5) In vivo use is more practical and safer due to lower concentrations needed and lack of neutralizing antibody production; 6) Tissue or cell specific gene targeting is possible using specific promoter vector (Paul, et al. 2002. Nat Biotechnol 20:505-508; Devroe, et al. 2002. BMC Biotechnol 2:15, each of which is incorporated by reference herein in its entirety) or specific antibody conjugated liposomes; 7) Simultaneously targeting multiple genes or multiple exons silencing is possible for increasing efficacy (Yang, et al. 2002. Proc Natl Acad Sci USA 99:9942-9947, which is incorporated by reference herein in its entirety).

### SUMMARY OF THE INVENTION

In general, disclosed are methods and compositions useful for modulating the immune system for induction of systemic anti-tumor responses subsequent to a localized release of tumor antigens precipitated by in situ tumor cell death.

Numerous solid tumors secrete a variety of immune suppressive factors in the local microenvironment (Botti, et al. 1998. Int J Biol Markers 13:51-69, which is incorporated by reference herein in its entirety), as well as induce secretion of immune suppressive factors from non-tumor cells in the microenvironment (Astigiano, et al. 2005. Neoplasia 7:390-396; Gilboa, E. 1999. Cancer Immunol Immunother 48:382-385; Yang, et al. 2004. Adv Cancer Res 92:13-27; Takahashi, et al. 2003. Int J Cancer 104:393-399, each of which is incorporated by reference herein in its entirety). Additionally, in cases such as the hepatic microenvironment, the natural milieu is intrinsically tolerogenic, even in healthy hosts (Sanchez-Fueyo, A. 2005. Gastroenterol Hepatol 28:250-256; Parker, et al. 2005. Toxicol Pathol 33:52-62, each of which is incorporated by reference herein in its entirety). Therefore when tumor death is induced by external means, such as, for example, chemotherapy, embolization, or radiotherapy, the antigens released in the local microenvironment do not appropriately prime immune responses for systemic effects to inhibit not only residual tumor growth, but also micrometastasis. In some situations, the release of antigens by radiation therapy can be used to prime immune responses by administration of exogenously generated syngeneic dendritic cells (DC) (Nikitina, et al. 2001. Int J Cancer 94:825-833; Teitz-Tennenbaum, et al. 2003. Cancer Res 63:8466-8475, each of which is incorporated by reference herein in its entirety), however this approach is not clinically practical due to extensive requirements for practicing cellular therapy. Further suggestion of the immunological relevance of localized tumor cell death is provided by evidence that in situ electromagnetic ablation of B16 tumors leads to induction of antigen-specific effectors that can be expanded by treatment with anti-CTLA4 antibodies (den Brok, et al. 2004. Cancer Res 64:4024-4029, which is incorporated by reference herein in its entirety). Additionally, anecdotal evidence exists of distant metastasis undergoing regression following localized application of cryoablation to the primary tumor cell mass (Soanes, et al. 1970. J Urol 104:154-159; Sanchez-Ortiz, et al. 2003. J Urol 170:178-179, each of which is incorporated by reference herein in its entirety). The cell-death inducing procedure of androgen ablation is prostate cancer patients has also been reported to associate with increased immune reactivity as a consequence of antigen release. Despite these observations, the use of augmentative immune therapy to enhance the potential immune-focusing effect of antigen release has not been described in a practical, clinically applicable manner (Mercader, et al. 2001. Proc Natl Acad Sci USA 98:14565-14570, which is incorporated by reference herein in its entirety).

The invention disclosed teaches that immunopotentiation at the local microenvironment during situations of tumor cell death can act as an “endogenous vaccine” for priming and promoting of systemic immunity.

In the first aspect a method of treating cancer is disclosed, comprising the localized administration of an iodinated oil mixture, with an immune stimulatory agent capable of reversing endogenous local and/or tumor-induced tolerogenic mechanisms together with an embolizing agent to a patient in need of therapy. The iodinated oil mixture could be the commonly used lipiodol solution, or novel derivatives thereof such as described in U.S. Pat. No. 6,690,962, which is incorporated by reference herein in its entirety. The immune stimulatory mixture could be a single agent capable of activating various immune cells, or could be a mixture of immune stimulators, or could be an agent capable of reversing immune suppression, or could be a mixture of agents that reverse immune suppression with agents that stimulate immune activation. The embolizing agent could be gelatin particles, or cyanoacrylate mixtures as described in U.S. Pat. No. 6,476,069, which is incorporated by reference herein in its entirety. Additionally the use of other agents that induce either tumor cell necrosis or apoptosis, such as chemotherapeutic, radiotherapeutic, or agents that synergize with the aforementioned therapies may also be used to enhance localized cell death and antigen release. One skilled in the art would be familiar with Ohmoto et al who demonstrated utility of electromagnetic ablation together with TACE as a means of synergistically achieving tumor necrosis (Ohmoto, et al. 2005. Hepatogastroenterology 52:1344-1346, which is incorporated by reference herein in its entirety). Furthermore, prior to the embolization, agents may be administered either locally or systemically to enhance the expression of tumor antigens, said agents could include sodium phenylbutyrate, trinchostatin A, or 5-azacytidine. The administration of the mixture could be sequentially, concurrently, or in cycles. One type of administration would be through performing the transcatheter embolization procedure in a patient with primary hepatic cancer.

Another aspect of the invention teaches methods of inducing anti-tumor immunity through augmenting the number of localized antigen presenting cells prior to induction of localized tumor cell death. Such augmentation of antigen presenting cell numbers can be accomplished by providing agents such as GM-CSF or flt-3L. Preferably the augmenter of antigen presenting cell numbers is maintained in the localized tumor environment prior to induction of tumor cell death. Said localization can be achieved through sponges, antibody targeting of the antigen presenting cell stimulatory molecules or, the extracellular matrix, or slow-release depot forming mixtures.

Another aspect of the invention teaches methods of inducing anti-tumor immunity through augmenting the cytokine secretion capability of cells from the tumor microenvironment such as fibroblasts, mesenchymal cells, endothelial and macrophages through administration of TLR agonists. In one embodiment TLR agonists are chosen for stimulation of Th1 cytokines such as IFN-γ, IL-12, and inhibition of suppressive cytokines such as IL-10 or TGF-β. Specific TLR agonists would have to be active on non-immune cells and could include agonists of TLR 1-12.

Another aspect of the invention teaches methods of inducing anti-tumor immunity through the sequential induction of tumor cell death followed by immune stimulation in a cyclical manner. Specifics of the time gap between cycles, as well as total number needed is based on a combination of regression responses seen, as well as immunological endpoints.

Another aspect of the invention teaches methods of inducing anti-tumor immunity through the use of lipiodol to transfect tumor cells, and cells of the tumor microenvironment with various types of short interfering RNA (siRNA) molecules for modification of the tumor cell environment. In one embodiment silencing of genes associated with immune suppression, such as IL-10, IDO, PD-1L, DAF, or CD55, in the tumor itself, and/or the microenvironment is used in order to render the tumor cell more sensitive to immunological attack.

Another aspect of the invention is the addition of immune stimuli to the TACE procedure when it is being performed in the extra-hepatic context, for example in lung metastasis as described by Shitaba et al. (Shibata, et al., e-published Sep. 16, 2005. Transcatheter Arterial Embolization for Tumor Seeding in the Chest Wall After Radiofrequency Ablation for Hepatocellular Carcinoma. Cardiovasc Intervent Radiol., which is incorporated by reference herein in its entirety).

Another aspect of the invention involves administration of an agent capable of reducing levels of complement inhibitors on tumor cells, such as sodium phenylbutyrate (Andoh, et al. 2002. Cancer Immunol Immunother 50:663-672, which is incorporated by reference herein in its entirety), prior to and/or subsequent to administration of either conventional TACE or TACE together with a local immune stimulant.

Another aspect of the invention discloses compositions of matter suitable for use in stimulation of localized immune response. Such compositions involve a stable depot of immune stimulators such as TLR agonists, which program the immunological microenvironment to present tumor antigens in an immunostimulatory fashion in order to allow for induction of systemic immunity.

Another aspect of the invention provides therapeutic kits for treatment of cancer comprising agents and compounds that used sequentially augment tumor immunity. In one embodiment, following induction of localized immune activation, the application of an immune stimulator capable of increasing memory cell turnover is administered. Said stimulator could be an agent which induces IL-15 such as imiquimod. In another embodiment, the process of lymphodepletion is induced prior to localized immune activation in order to initiate a process of homeostatic expansion in the presence of localized antigen release.

Another aspect of the invention provides methods of therapeutic monitoring of the immunity stimulated in the microenvironment such that either cycles of immunization or additional adjuvant therapies can be administered to increase efficacy. In one embodiment the monitoring is performed by analysis of the patient T cells of immune activation in response to either tumor cell lysates, defined protein antigen, or tumor derived peptides. Responses may include but are not limited to cytokine production, expression of memory T cell phenotype, as well as functional proliferative and cytotoxic activity.

In an embodiment of the present invention, a method of inducing an anticancer immune response in a cancer patient is provided, by admixing a concentration of immune stimulant with a clinically applicable localizing agent and a single or plurality of agents capable of causing localized cell death, administering the combination directly into the tumor and/or arteries providing the tumor with blood supply, and administering an embolizing agent in the proximity of the tumor and/or directly into the arteries providing the tumor with blood supply. The immune stimulant can be, for example, a small molecule, a nucleic acid, a protein, or a combination thereof. The small molecule immune stimulant can be selected from, for example, muramyl dipeptide, thymosin, 7,8-disubstituted guanosine, imiquimod, detoxified lipopolysaccharide, isatoribine and alpha-galactosylceramide. The nucleic acid can be selected from, for example, a short interfering RNA targeting the mRNA of immune suppressive proteins, CpG oligonucleotides, Poly IC, unmethylated oligonucleotides, plasmid encoding immune stimulatory molecules, or chromatin-purified DNA. The protein can be selected from, for example, IL-2, IL-7, IL-8, IL-12, IL-15, IL-18, IL-21, IL-23, IFN-α, β, γ, TRANCE, TAG-7, CEL-1000, bacterial cell wall complexes, or LIGHT. The agent capable of causing cell death can be a chemotherapeutic or radiotherapeutic agent. The localizing agent can be, for example, an iodinated oil mixture. The localizing agent can be, for example, lipiodol. The embolizing agent can be, for example, Avitene, Gelfoam, Occlusin or Angiostat.

In another embodiment of the present invention, a method of inducing an anticancer immune response to a patient in need thereof is provided, by admixing a concentration of short interfering RNA with a clinically applicable localizing agent and a single or plurality of agents capable of causing localized cell death, administering the combination directly into the tumor and/or arteries providing the tumor with blood supply; and administering an embolizing agent in the proximity of the tumor and/or directly into the arteries providing the tumor with blood supply. The short interfering RNA can be administered, for example, in the one of the following forms: DNA plasmids capable of transcribing hairpin loop RNA which is subsequently cleaved by endogenous cellular processes into short interfering RNA, double stranded RNA chemically synthesized oligonucleotides, in vitro generated siRNA fragments from mRNA. The short interfering RNA can be targeted, for example, to one or more mRNA selected from the following group: IDO, IL-4, IL-10, TGF-β, FGF, and VEGF. The cell death can be caused, for example, by a chemotherapeutic or radiotherapeutic agent, or by embolization of the tumor.

In a further embodiment of the present invention, a pharmaceutical composition capable of eliciting an antigen-specific immune response to tumor derived proteins is provided, having an immune stimulant, a clinically applicable localizing agent, an agent capable of causing cell death, and an embolizing agent. The immune stimulant can be, for example, a small molecule, a nucleic acid, a protein, or a combination thereof. The small molecule immune stimulant can be selected from, for example, muramyl dipeptide, thymosin, 7,8-disubstituted guanosine, imiquimod, detoxified lipopolysaccharide, isatoribine or alpha-galactosylceramide. The nucleic acid can be selected from, for example, short interfering RNA targeting the mRNA of immune suppressive proteins, CpG oligonucleotides, Poly IC, unmethylated oligonucleotides, plasmid encoding immune stimulatory molecules, and chromatin-purified DNA. The protein can be selected from, for example, IL-2, IL-7, IL-8, IL-12, IL-15, IL-18, IL-21, IL-23, IFN-α, β, γ, TRANCE, TAG-7, CEL-1000, bacterial cell wall complexes, or LIGHT. The agent capable of causing cell death can be a chemotherapeutic or radiotherapeutic agent. The cell death can be caused, for example, by embolization of the tumor with an embolizing agent that is selected from Avitene, Gelfoam and Angiostat.

In a further embodiment of the present invention, a method of modification of the transcatheter chemoembolization procedure is provided, in order to induce an antitumor immune response to in a patient with hepatic cancer in need thereof, by selecting a patient suitable for therapy, inserting a catheter into the patient, administering a mixture of a single or plurality of immune stimulant(s) admixed with a clinically applicable localizing agent and/or with a single or plurality of agents capable of causing localized cell death, administering the combination directly into the tumor and/or arteries providing the tumor with blood supply using said catheter, administering an embolizing agent in the proximity of the tumor and/or directly into the arteries providing the tumor with blood supply using said catheter, assessing the levels of immune activation, providing subsequent agents to enhance/maintain immune activation, and performing the procedures of the above steps as needed determined by the level of immune activation and/or tumor regression. The patient can meet the current standard of care inclusion/exclusion criteria for eligibility for transcatheter chemoembolization. The patient can suffer from, for example, a localized primary hepatocellular carcinoma, or a hepatically-located metastasis originating from a tumor exterior to the liver. The tumor can be, for example, a functional neuroendocrine cancer such as a carcinoid tumor or a pancreatic endocrine tumor. In some embodiments, the cancer patient has failed systemic therapy with octreotide to control carcinoid syndrome. The tumor can be, for example, unresectable, or tumor growth control can be desired until a liver transplant is feasible. The patient can have adequate hepatic function as determined by a plasma concentration of bilirubin <2 mg/dl; plasma albumin of >2.7g/dl; and no portal vein occlusion. The patient can have, for example, adequate renal function as determined by plasma concentration of creatinine <2 mg/dl. The catheter can be inserted using the Seldinger technique, and passed under fluoroscopic control into the hepatic artery determined to be the tumor feeding artery. The mixture injected into the tumor feeding artery can have, for example, a composition of Poly (IC), lipiodol, and doxorubicin at a concentration sufficient to induce localized tumor cell death, immune activation, and form a localized depot. The mixture injected into the tumor feeding artery can be, for example, a composition of an immune stimulant, lipiodol, and a chemotherapeutic agent at a concentration sufficient to induce localized tumor cell death, immune activation, and can form a localized depot. The immune stimulant can be capable of activating expression of immune stimulatory molecules on cells of the localized microenvironment. The chemotherapeutic agent can be capable of activating expression of immune stimulatory molecules on cells of the localized microenvironment. The chemotherapeutic agent can be, for example, melphalan. The chemotherapeutic agent can be capable of upregulating antigenic expression of tumor cells. The chemotherapeutic agent can be selected from 5-azacytidine, sodium phenylbutyrate, and trinchostatin A. The immune stimulant can be a protein, or a combination thereof. The small molecule immune stimulant can be selected from muramyl dipeptide, thymosin, 7,8-disubstituted guanosine, imiquimod, detoxified lipopolysaccharide, isatoribine or alpha-galactosylceramide. The nucleic acid can be selected from a short interfering RNA targeting the mRNA of immune suppressive proteins, a CpG oligonucleotide, Poly IC, an unmethylated oligonucleotide, a plasmid encoding immune stimulatory molecules, or chromatin-purified DNA. The protein can be selected from, for example, IL-2, IL-7, IL-8, IL-12, IL-15, IL-18, IL-21, IL-23, IFN-α, β, γ, TRANCE, TAG-7, CEL-1000, bacterial cell wall complexes, or LIGHT. The short interfering RNA can be administered in the one of the following forms: DNA plasmids capable of transcribing hairpin loop RNA which is subsequently cleaved by endogenous cellular processes into short interfering RNA, double stranded RNA chemically synthesized oligonucleotides, in vitro generated siRNA fragments from mRNA. The short interfering RNA can be targeted, for example, to one or more mRNA selected from IDO, IL-4, IL-10, TGF-β, FGF, or VEGF. The embolizing agent can be selected from, for example, Avitene, Gelfoam, Occlusin and Angiostat. The cell death causing agent can be a radiotherapeutic. The cell death can be caused or accelerated from a group of therapeutic approaches such as radiofrequency ablation, localized hyperthermia, conformal radiotherapy, and antibody-target radiotherapeutics. The immune activation state can be assessed, for example, in an antigen-specific or non-antigen-specific manner. The antigen-specific immune activation can be quantitated by the numbers of tetramer positive T cells identified by staining with a tetramer bearing a tumor antigen. The antigen can be specific for liver carcinoma. The antigen can be selected from, for example, a MAGE peptide, an NY-ESO-1b peptide, and an alpha-fetoprotein derived peptide. The T cells can be tetramer positive and can express interferon gamma spontaneously or upon ex vivo restimulation. The T cells can be examined for expression of function and cleaved T Cell Receptor zeta-chain. The T cells can be examined for the ability to proliferate ex vivo in response to antigen challenge. The antigen specific immune response can be assessed, for example, by the ability of the patient immune response to form a delayed type hypersensitivity reaction to antigenic sources selected from an autologous tumor cell lysate, an allogeneic tumor cell lysate, a MAGE peptide, an NY-ESO-1b peptide, and an alpha-fetoprotein derived peptide. A model antigen such as ovalbumin or keyhole limpet hemocyanin can be originally administered as part of the chemoembolization mixture, and immune response to it can be assessed by methods selected from, for example, tetramer positivity for the antigen, expression of functional TCR zeta chain on tetramer positive cells for the antigen, proliferative response to the antigen ex vivo, cytokine production ability in response to the antigen ex vivo, and the ability to generate delayed type hypersensitivity reactions to the antigen. The T cell memory formation in response to the antigens and antigenic compositions can be assessed, for example, by expression of markers associated with either T cell central memory or T cell effector memory phenotypes. The T cell central memory cells can be, for example, positive for expression of CD45RO, CCR7 whereas T cell effector memory cells are positive for expression of CD45RO and negative for expression of CCR7. The immune response can be assessed, for example, through assaying non-antigen specific measurements of immune activation selected from T cell proliferative, cytokine, and activation marker responses to ex vivo stimuli such as conconavalin A, phytohemagglutinin, anti-CD3 together with anti-CD28. The non-antigen specific immune activation can be assessed, for example, by the cytokine release and natural cytotoxicity of natural killer, and/or natural killer T cells. The antibody mediated responses can be assessed, for example, as a measure of antigen-specific immune stimulation. The dose of an agent capable of maintaining/augmenting an immune response can be administered at a timepoint and concentration sufficient to increase immunological control of the neoplasia. The agent capable of increasing immune response can be administered, for example, prior to the embolization procedure. The agent can be a T cell depleting agent administered in such as manner so as to cause a state of homeostatic lyphoproliferative expansion before the embolization procedure. The agent being selected from, for example, radiotherapy, cyclophosphamide, Campath, and anti-CD3. The tumor antigen can be performed, for example, subsequent to the embolization procedure. The subsequent embolization procedure can be the method of tumor immunization. The agent capable of inducing memory cell turnover can be administered for immune stimulation. The agent can be selected from, for example, IFN-alpha, IL-12, IL-15, IL-18, and IL-23. The agent capable of inducing expression of cytokines selected from, for example, IFN-alpha, IL-12, IL-15, IL-18, and IL-23 can be administered. The agent can be, for example, an agonist of a toll-like receptor. The agent can be, for example, imiquimod.

In a further embodiment of the present invention, a method of altering the hepatic microenvironment as to make it inhospitable for tumor growth is provided, by introducing into the hepatic microenvironment an agent capable of immune stimulation, concurrently adding a localizing agent, and adjusting the dose based on immunological parameters known in the art to prevent engraftment of metastatic tumors.

In a yet further embodiment of the present invention, a method of preconditioning the liver microenvironment prior to induction of localized tumor cell death is provided, so as to enhance the ability of the immune response to induce anti-tumor effectors subsequent to induction of tumor cell death. The preconditioning is achieved through activation of hepatic natural killer t cells. The activation of natural killer T cells can be accomplished, for example, through administration of an agent that indirectly induces activation of said natural killer T cells through stimulating production of activitory compounds by hepatic dendritic cells. The agent can be selected from Poly IC, muramyl dipeptide, thymosin, 7,8-disubstituted guanosine, imiquimod, detoxified lipopolysaccharide, isatoribine or alpha-galactosylceramide. Prior to administration of a dendritic cell activator, the dendritic cell numbers can be enhanced through supplying an effective amount of DC progenitor proliferative stimuli. The DC progenitor proliferative stimuli can be selected from, for example, fins-like tyrosine kinase-3 ligand, GM-CSF, progenipoietin-1, and thrompoietin.

In an additional embodiment of the present invention, a method of immune modulating the systemic host prior to induction of tumor cell death is provided, in order to enhance the ability of the immune response to induce anti-tumor effectors subsequent to induction of localized tumor cell death. The systemic repair of T cell abnormalities can be accomplished, for example, through administration of a sufficient dose of anti-oxidants selected from n-acetylcysteine, ascorbic acid, genistein, co-enzyme Q-10, alpha lipoic acid, and vitamin E. An agent capable of reducing the activation threshold necessary for T cell activation can be added. The agent can be selected, for example, from an antagonistic anti-CTLA-4 antibody, an agonisting anti-CD28 antibody, a depleting anti-CD25 antibody, a low dose IL-2, and a TLR agonist.

In an additional embodiment of the present invention, a method of systemically immune modulating the host subsequent to induction of localized tumor cell death is provided, so as to enhance the ability of the immune response to induce anti-tumor effectors subsequent to induction of localized tumor cell death. The systemic repair of T cell abnormalities can be accomplished, for example, through administration of a sufficient dose of anti-oxidants selected from n-acetylcysteine, ascorbic acid, genistein, co-enzyme Q-10, alpha lipoic acid, and vitamin E. An agent capable of reducing the activation threshold necessary for T cell activation can be added. The agent can be selected from, for example, an antagonistic anti-CTLA-4 antibody, an agonisting anti-CD28 antibody, a depleting anti-CD25 antibody, low dose IL-2, and a TLR agonist.

In an additional embodiment of the present invention, a method of effecting immune modulation in a host in need thereof is provided, by administration of short interfering RNA in a composition of lipiodol. The siRNA can be administered, for example, in the form of a therapeutic vaccine in combination with an adjuvant. The adjuvant can be selected from QS-21, complete Freund's adjuvant, incomplete Freund's adjuvant, agonistic anti-CD40 antibody, Montanide ISA-51, and IL-12. The adjuvant can be a TLR agonist. The TLR agonist can be imiquimod. The siRNA hybridizes with the transcript of an immune suppressive molecule. The immune suppressive molecule can be selected from, for example, IL-10, TGF-β, Fas ligand, VEGF, IL-18 binding protein, decoy receptor 3, heavy chain ferritin and protectin/CD59. The siRNA can be administered at a concentration sufficient to induce the process of RNA interference. siRNA can be administered in a composition of lipiodol, with procedures and compositions known to induce necrosis of tumor cells. Procedures can be selected from a transcatheter chemoembolization, transcatheter embolization, radiofrequency ablation, localized hyperthermia, conformal radiotherapy, and antibody-target radiotherapeutics.

Advantages of the various aspects and embodiments of the present invention include: lack of systemic toxicity, augmentation of endogenous natural tumor suppressive mechanisms, as well as methods of monitoring the immune status of the patient in order to provide individualized therapy.

The foregoing has overviewed in a rather broad fashion the features and specific advantages of the present invention in order that the detailed description of the invention that follows may be better understood. Further specifics and methods of practicing the invention will be described afterwards, which comprise the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent embodiments or manifestations do not depart from the spirit and scope of the invention as set forth in the appended claims. The novel features which are believed to be characteristic of the invention, both as to its organization and method of operation, together with further objects and advantages will be better understood from the following description when considered in connection with the accompanying examples and the current state-of-the-art. It is to be understood, however, that each of the figures is provided for the purpose of illustration and description only and is not intended as a definition of the limits of the present invention.

### BRIEF DESCRIPTION OF THE DRAWINGS

FIG. 1. Intrahepatic administration of Lipiodol/Poly (I:C) augments OVA-specific proliferative responses.

FIG. 2. Intrahepatic administration of Lipiodol/Poly (I:C) augments OVA-specific IFN-γ responses.

FIG. 3. Intrahepatic administration of Lipiodol/Poly (I:C) augments OVA-specific DTH responses.

FIG. 4. Intrahepatic administration of Lipiodol/Poly (I:C) alleviates need for boosting of OVA-specific proliferative responses.

FIG. 5. Intrahepatic administration of Lipiodol/Poly (I:C) alleviates need for boosting of OVA-specific IFN-γ responses.

FIG. 6. Intrahepatic administration of Lipiodol/Poly (I:C) alleviates need for boosting of OVA-specific DTH responses.

FIG. 7. Lipiodol can act as a siRNA transfection reaction to purified DC.

FIG. 8. Lipiodol-siRNA transfection leads to DC cytokine immune modulation.

FIG. 9. Lipiodol-siRNA transfection leads to altered DC allostimulatory ability.

FIG. 10. Lipiodol-siRNA transfection leads to altered DC cytokine-evoking ability (IFN-γ).

FIG. 11. Lipiodol-siRNA transfection leads to altered DC cytokine-evoking ability (IL-4).

FIG. 12. Lipiodol-siRNA vaccination alters systemic T Cell proliferative responses.

FIG. 13. Lipiodol-siRNA vaccination alters systemic T Cell IFN-γ responses.

FIG. 14. Lipiodol-siRNA vaccination alters systemic T Cell IL-4 responses.

FIG. 15. Immune stimulation by Lipiodol-siRNA targeting IL-10: Proliferative Recall.

FIG. 16. Immune stimulation by Lipiodol-siRNA targeting IL-10: Increased IFN-γ.

FIG. 17. Immune stimulation by Lipiodol-siRNA targeting IL-10: Decreased IL-4.

### DETAILED DESCRIPTION

Without intending to be limited by theory, the invention disclosed teaches methods of utilizing the immune response of a cancer patient in a therapeutic manner to control tumor recurrence and/or metastasis subsequent to a procedure during which tumor antigens are released.

Numerous procedures are clinically used that are associated with release of tumor antigens. Especially attractive procedures to which this invention is tailored are procedures associated with induction of tumor cell necrosis in a localized microenvironment. Specifically therapies such as transcatheter chemoembolization (TACE) conformal radiotherapy, percutaneous ethanol administration, embolization therapy, localized hyperthermia, and electromagnetic ablation therapy.

One specific embodiment of the invention involves modification of the TACE procedure in order to induce a systemic anti-tumor immunological effect. Specifically, patients are selected to meet the criteria for TACE. Said criteria includes: a) Adequate hepatic function; b) Patent portal vein circulation (confirmed during the venous phase of celiac or superior mesenteric angiogram); and c) Adequate renal function. Generally, only patients without cirrhosis or in Child group A or B disease are considered, however depending on experience of the practicing physician other groups may be included in the procedure as discussed by Shah et al. (Shah, et al. 1998. Qjm 91:821-828, which is incorporated by reference herein in its entirety). The TACE procedure may be performed either using a selective or superselective means. Patients selected to undergo the procedure receive 10 mg of phytonadione intravenously prior to the procedure (the intravenous injection should be administered slowly). Femoral catheterization and positioning of the catheter is performed. Premedication is with Lorazepam (Wyeth Laboratories, UK) 0.25 mg/kg orally 1 hour before the procedure to counter anxiety. An intra-arterial injection of 30-40 mg of 1% lidocaine is used for analgesia.

The following ingredients are made into an emulsion by repeatedly emptying and filling a syringe over 10 minutes: 10 mL of Lipiodol Ultrafluid (Mallinckrodt Medical, UK), 5 mL Omnipaque 300 (Amersham Health, UK; water-soluble contrast aids in emulsifying the mixture), 50 mg doxorubicin and clinical grade Poly (IC) stabilized with carboxymethylcellulose at a concentration between 0.025 mg/m² to 12 mg/m², preferably at a concentration of 0.2 mg/m². Intraarterial injection is administered under direct visualization to prevent reflux into gastroduodenal or splenic vessels. Embolization is performed with Ultra Ivalon 250-400 μm (Laboratories Nyrcomed SA). Intravenous cefuroxime (750 mg) and metronidazole (500 mg) are administered 3 times per day for 5 days. These antibiotics are given as prophylaxis against septicemia and liver abscess formation. Subsequent to administration patients are admitted to a high-dependency ward and should be mobilized after 6 hours of bedrest. Postoperative analgesia is administered if and when required by the patient. Patients also receive ranitidine (an H2 antagonist) intravenously 3 times per day until they begin eating. Patients are discharged home after 5 days or when their systemic symptoms begin resolving.

In order to monitor success of the procedure nonenhanced and enhanced CT examinations are performed 10-14 days following embolization. Furthermore, alpha-fetoprotein levels are evaluated at the 6-week outpatient review. If the TACE procedure is successful (>50% lipiodol uptake in necrotic tumor demonstrated on the postprocedural CT scan), the embolization is repeated in 6-8 weeks. Immunological monitoring is performed by assessing levels of interferon alpha production using ELISA during the 12, 24, and 72 hour time periods. Additionally, DTH, cellular and antibody responses are measured using pre-defined antigens representative of the tumor type.

A variety of chemotherapeutic agents can be used in practicing the invention. Specifically, chemotherapeutic agents which induce upregulation of costimulatory molecules are preferred. One example of such an agent is melphalan, which induces expression of CD80 on both tumor cells (Donepudi, et al. 2001. J Immunol 166:6491-6499, which is incorporated by reference herein in its entirety), as well as non-tumor B cells (Donepudi, et al. 2003. Cancer Immunol Immunother 52:162-170, which is incorporated by reference herein in its entirety). In addition, a wide variety of chemotherapeutic agents are known in the art. These include, but are not limited to, alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; arnsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid; esperamicins; capecitabine, and the like.

The above mentioned TACE-modification procedure was presented as an example of illustrating the disclosed invention. Additionally modifications may be made to increase efficacy of anti-tumor response being mediated. Particularly, a wide variety of agents can be administered to the patient prior to the TACE procedure in order to increase general immunological status, and specifically, T cell, NK cell, and NKT cell functions. One particular modification may involve the administration of an anti-oxidant capable of reversing immune suppression seen in many cancer patients. Immune suppression by cancer has been well-documented in advanced cancer patients possessing a variety of malignancies (Ng, et al. 2002. Urology 59:9-14; Campbell, et al. 2001. J Immunol 167:553-561; Beck, et al. 2001. Microsc Res Tech 52:387-395; Almand, et al. 2001. J Immunol 166:678-689; Dix, et al. 1999. J Neuroimmunol 100:216-232; Kiessling, et al. 1999. Cancer Immunol Immunother 48:353-362; Kim, et al. 1999. J Korean Med Sci 14:299-303; Ungefroren, et al. 1999. Ann NY Acad Sci 880:243-251, each of which is incorporated by reference herein in its entirety). Correlation between immune suppression and poor prognosis has been extensively noted (Fischer, et al. 1997. Ann Oncol 8:457-461; Ishigami, et al. 2002. Cancer 94:1437-1442; Marana, et al. 2000. Int J Gynecol Cancer 10:67-73, each of which is incorporated by reference herein in its entirety). Several means of tumor suppression of immune response are known. For example, a variety of tumor cells possess the ability to induce cleavage of the T cell receptor zeta (TCR-ζ) chain through a caspase-3 dependent manner (Gastman, et al. 2000. Blood 95:2015-2023; Takahashi, et al. 2001. Clin Cancer Res 7:74-80, each of which is incorporated by reference herein in its entirety). Since TCR-ζ is critical for signal transduction, host T cells become unable to respond to tumor antigens. Originally, the TCR-ζ cleavage was described in tumor bearing mice (Mizoguchi, et al. 1992. Science 258:1795-1798; Horiguchi, et al. 1999. Cancer Res 59:2950-2956, each of which is incorporated by reference herein in its entirety) and subsequently in patients (Schmielau, et al. 2001. Cancer Res 61:4756-4760; Kim, et al. 1999. Pathobiology 67:123-128; Laytragoon-Lewin, et al. 2000. Anticancer Res 20:1093-1100; Taylor, et al. 2001. Br J Cancer 84:1624-1629; Chen, et al. 2000. Br J Haematol 111:817-825; Healy, et al. 1998. Cytometry 32:109-119, each of which is incorporated by reference herein in its entirety). The correlation between suppressed TCR-ζ and suppressed IFN-γ production has been reported, implying functional consequences (Kim, et al. 1999. Pathobiology 67:123-128, which is incorporated by reference herein in its entirety). The cause of TCR-ζ suppression has been attributed, at least in part, to reactive oxygen radicals produced by: A) The inflammatory activity occurring inside the tumor (it is well established that there is a constant area of necrosis intratumorally; B) Macrophages associated with the tumor; and C) Neutrophils activated directly by the tumor, or by the tumor associated macrophages.

Tumors are usually associated with macrophage infiltration, this is correlated with tumor stage and is believed to contribute to tumor progression by stimulation of angiogenesis (Valkovic, et al. 2002. Virchows Arch 440:583-588; Makitie, et al. 2001. Invest Ophthalmol Vis Sci 42:1414-1421; Leek, et al. 1996. Cancer Res 56:4625-4629, each of which is incorporated by reference herein in its entirety). Cytokines such as M-CSF (Valkovic, et al. 2002. Virchows Arch 440:583-588, which is incorporated by reference herein in its entirety) and VEGF (Lewis, et al. 2000. J Pathol 192:150-158, which is incorporated by reference herein in its entirety) produced by tumor infiltrating macrophages are essential for tumor progression to malignancy. In fact, tumors implanted into M-CSF deficient op/op mice (that lack macrophages) do not metastasize or become vascularized (Nowicki, et al. 1996. Int J Cancer 65:112-119, which is incorporated by reference herein in its entirety). Tumor-associated macrophages possess an activated phenotype and release various inflammatory mediators such as cyclo-oxygenase metabolites (Kamate, et al. 2002. Int J Cancer 100:571-579; Young, et al. 1987. J Leukoc Biol 42:682-688, each of which is incorporated by reference herein in its entirety), TNF-α (Billingsley, et al. 1996. Ann Surg Oncol 3:29-35, which is incorporated by reference herein in its entirety), and IL-6 (Bonta, et al. 1993. J Leukoc Biol 54:613-626, which lead to increased levels of oxidative stress produced by host immune cells. In addition, tumor associated macrophages themselves produce large amounts of free radicals such as NO, OH, and H₂O₂ (Bhaumik, et al. 1998. Nitric Oxide 2:467-474; Lewis, et al. 1987. Environ Health Perspect 76:19-27; Kono, et al. 1996. Eur J Immunol 26:1308-1313, each of which is incorporated by reference herein in its entirety). The high levels of macrophage activation in cancer patients is illustrated by high serum levels of neopterin, a tryptophan metabolite that is associated with poor prognosis (Murr, et al. 2002. Curr Drug Metab 3:175-187, which is incorporated by reference herein in its entirety). In addition to oxidative stress elaborated by tumor associated macrophages, the presence of the tumor itself causes systemic changes associated with chronic inflammation. Erythrocyte sedimentation ration, C-reactive protein and IL-6 are markers of inflammatory stress used to designate progression of pathological immune diseases such as arthritis (Whisler, et al. 2002. Clin Podiatr Med Surg 19:149-161, vii; Ishihara, et al. 2002. Cytokine Growth Factor Rev 13:357, each of which is incorporated by reference herein in its entirety). Interestingly advanced cancer patients possess all of these inflammatory markers (Mahmoud, et al. 2002. Curr Oncol Rep 4:250-255; Smith, et al. 2001. Cytokine Growth Factor Rev 12:33-40; Rutkowski, et al. 2002. Int J Cancer 100:463-471; Kallio, et al. 2001. J Exp Clin Cancer Res 20:523-528; Ljungberg, et al. 1997. Eur J Cancer 33:1794-1798, each of which is incorporated by reference herein in its entirety). Another marker of chronic inflammation is decreased albumin synthesis by the liver, this is also seen in cancer patients and is believed to contribute, at least in part, to cachexia (Fearon, et al. 1999. World J Surg 23:584-588; McMillan, et al. 2001. Nutr Cancer 39:210-213, each of which is incorporated by reference herein in its entirety). In addition, the inflammatory marker fibrinogen D-dimers is also higher in cancer patients as opposed to controls (Oya, et al. 2001. Jpn J Clin Oncol 31:388-394; Ferrigno, et al. 2001. Eur Respir J 17:667-673; Blackwell, et al. 2000. J Clin Oncol 18:600-608, each of which is incorporated by reference herein in its entirety). Schmielau et al reported that in patients with a variety of cancers, activated neutrophils are circulating in large numbers (Schmielau, et al. 2001. Cancer Res 61:4756-4760, which is incorporated by reference herein in its entirety). These neutrophils secrete reactive oxygen radicals such as hydrogen peroxide, which trigger suppression of TCR-ζ and IFN-γ production. This was demonstrated by co-incubation of the neutrophils from cancer patients with lymphocytes from healthy volunteer. A profound suppression of TCR-ζ expression was seen. Evidence for the critical role of hydrogen peroxide was shown by the fact that addition of catalase suppressed TCR-ζ downregulation. A simple method of assessing the number of circulating activated neutrophils was described in the same paper. This method involves collecting peripheral blood from patients, spinning the blood on a density gradient such as Ficoll, and collecting the lymphocyte fraction. While in healthy volunteers the lymphocyte fraction contained primarily lymphocytes, in cancer patients the lymphocyte fraction contained both lymphocytes and a large number of neutrophils. The reason why these neutrophils are present in the lymphocyte fraction is because activation alters their density so that they co-purify differently on the gradient. A potential indication of the importance of activated neutrophils to cancer progression is provided by Tabuchi et al who show that removal of granulocytes from the peripheral blood of cancer patients resulted in reduced tumor size, unfortunately, the study was performed in only 2 patients (Tabuchi, et al. 1999. Cancer Detect Prev 23:417-421, which is incorporated by reference herein in its entirety). As a mechanism to compensate for immune over-activation, mediators of inflammation have immune suppressive properties. This is best illustrated in the immune suppression seen following immune hyperactivation such as in septic shock. Following the primary scepticemia, patients are systemically immune compromised due to circulating immune suppressive factors that are released in response to the inflammatory stress. This suppression is termed compensatory anti-inflammatory response syndrome (CARS) and is associated with many opportunistic infections and deactivation (Oberholzer, et al. 2001. Shock 16:83-96, which is incorporated by reference herein in its entirety). The clinical importance of CARS immune suppression is seen in that sepsis survivors show normal T-cell proliferation and IL-2 release, whereas those that succumb possess suppressed T cell responses (Heidecke, et al. 1999. Am J Surg 178:288-292., which is incorporated by reference herein in its entirety). Interestingly immune suppressive mediators associated with CARS such as PGE2, TGF-β, and IL-10 are also associated with cancer-induced immune suppression (Elgert, et al. 1998. J Leukoc Biol 64:275-290, which is incorporated by reference herein in its entirety). The role of oxidative stress in sepsis-induced immune suppression was recently demonstrated in experiments where administration of antioxidants (ascorbic acid or n-acetylcysteine) to animals undergoing experimental sepsis blocked immune suppression (De la Fuente, et al. 2001. Free Radic Res 35:73-84, which is incorporated by reference herein in its entirety). Another example of the potential for antioxidants to stimulate immune response in an inflammatory condition is in patients with Duke's C and D colorectal cancer who were administered of a daily dose of 750 mg of vitamin E for 2 weeks. This resulted in restoration of IFN-γ and IL-2 production (Malmberg, et al. 2002. Clin Cancer Res 8:1772-1778, which is incorporated by reference herein in its entirety). The problem of uncontrolled inflammation is seen in sepsis. Although as a monotherapy n-acetylcysteine has little clinical effect, therapeutic administration of n-acetylcysteine results in suppression of the constitutively activated neutrophils seen in these patients (Heller, et al. 2001. Crit Care Med 29:272-276, which is incorporated by reference herein in its entirety). Administration of n-acetylcysteine to smokers results in suppression of markers of oxidative stress (Van Schooten, et al. 2002. Cancer Epidemiol Biomarkers Prev 11:167-175, which is incorporated by reference herein in its entirety). Furthermore, oral n-acetylcysteine administration blocks angiogenesis and suppresses growth of Kaposi Sarcoma (Albini, et al. 2001. Cancer Res 61:8171-8178, which is incorporated by reference herein in its entirety). Accordingly, a method of preparing the host for the TACE procedure includes administration of n-acetylcysteine at a concentration sufficient to decrease the tumor associated suppression of T cell activity. Such a concentration ranges between 1-10 grams per day, preferably 4-6 grams administered intravenously for a period of type sufficient to normalize production of IFN-γ from PBMC of cancer patients upon ex vivo stimulation. One skilled in the art will understand that n-acetylcysteine is just one example of a compound suitable for reversion of oxidative-stress associated immune suppression. Numerous other compounds may be used, for example ascorbic acid (Leibovitz, et al. 1978. Int J Vitam Nutr Res 48:159-164; Siegel, et al. 1974. Infect Immun 10:409-410; Riordan, et al. 2003. P R Health Sci J 22:287-290, each of which is incorporated by reference herein in its entirety), co-enzyme Q10 in combination with vitamin E and alpha-lipoic acid (Folkers, et al. 1985. Drugs Exp Clin Res 11:539-545, which is incorporated by reference herein in its entirety), genistein (Ravindranath, et al. 2004. Adv Exp Med Biol 546:121-165, which is incorporated by reference herein in its entirety) or resveratrol (Li, et al. 2005. Xi Bao Yu Fen Zi Mian Yi Xue Za Zhi 21:575-579, which is incorporated by reference herein in its entirety).

CD4⁺ CD25⁺ T regulatory cells (Treg) are considered to be a “mirror-immune system” capable of recognizing a similar repertoire of antigens as conventional T cells, with the exception that instead of inducing immune activation, they suppress it (Lan, et al. 2005. Autoimmun Rev 4:351-363, which is incorporated by reference herein in its entirety). Treg cells are generated in the thymus by positive selection to self antigens, whereas conventional T cells are deleted intrathymically upon recognition of self antigens (Anderson, et al. 2005. Immunity 23:227-239, which is incorporated by reference herein in its entirety). Specifically, the Hassall's corposucle of the thymus was demonstrated to be the site of self-antigen reactive Treg generation (Watanabe, et al. 2005. Nature 436:1181-1185, which is incorporated by reference herein in its entirety). Additionally, Treg cells are generated in the periphery in response to self antigens being presented on tolerogenic or immature dendritic cells in the basal state or in situations of tolerance induction (Min, et al. 2003. J Immunol 170:1304-1312, which is incorporated by reference herein in its entirety). Treg cells are capable of suppressing T helper (Stassen, et al. 2004. Transplantation 77:S23-25, which is incorporated by reference herein in its entirety), T cytotoxic (Green, et al. 2003. Proc Natl Acad Sci USA 100:10878-1088, which is incorporated by reference herein in its entirety), T memory (Levings, et al. 2001. J Exp Med 193:1295-1302, which is incorporated by reference herein in its entirety), and NKT cell function (Azuma, et al. 2003. Cancer Res 63:4516-4520, which is incorporated by reference herein in its entirety), as well as ability of DC to mature (Serra, et al. 2003. Immunity 19:877-889, which is incorporated by reference herein in its entirety) through a variety of mechanisms including surface bound TGF-β (Huber, et al. 2006. Front Biosci 11:1014-1023, which is incorporated by reference herein in its entirety), granzyme B secretion (Gondek, et al. 2005. J Immunol 174:1783-1786, which is incorporated by reference herein in its entirety), and IL-10 release (Mekala, et al. 2005. Proc Natl Acad Sci USA 102:11817-11822, which is incorporated by reference herein in its entirety).

The immune regulatory role of Treg is demonstrated by ability to accelerate collagen induced arthritis when these cells are depleted using anti-CD25 antibodies (Morgan, et al. 2003. Arthritis Rheum 48:1452-1460, which is incorporated by reference herein in its entirety). Additionally, the potency of tumor vaccines is also known to increase following Treg depletion (Nagai, et al. 2004. Exp Dermatol 13:613-620, which is incorporated by reference herein in its entirety). Clinical trials using the CD25⁺ T cell-depleting agent ONTAK which delivers a dose of diphteria toxin to CD25⁺ cells, have demonstrated improved cellular immunity following RNA-DC vaccination (Dannull, et al. 2005. J Clin Invest. 115(12):3623-3633, which is incorporated by reference herein in its entirety). Additional support for the role of Treg in maintaining tolerance comes from experimentally situations whereby tolerance induction does not occur in hosts deficient in Tregs. This was demonstrated in the transplantation tolerance (Banuelos, et al. 2004. Transplantation 78:660-667; Taylor, et al. 2001. J Exp Med 193:1311-1318, each of which is incorporated by reference herein in its entirety), oral tolerance (Dubois, et al. 2003. Blood 102:3295-3301, which is incorporated by reference herein in its entirety), and altered peptide ligand (Yamashiro, et al. 2002. Int Immunol 14:857-865, which is incorporated by reference herein in its entirety) models of tolerogenesis.

Clinically, conditions of tolerance are associated with Treg cell activity. Specifically, in patients with rheumatoid arthritis a reduction in Treg activity was demonstrated. Interestingly, the administration of anti-TNF-alpha antibodies lead to increased Treg activity in patients clinically responding (Ehrenstein, et al. 2004. J Exp Med 200:277-285, which is incorporated by reference herein in its entirety). A reduction in the enhanced rate of spontaneous Treg apoptosis caused by anti-TNF-alpha antibody was postulated as a mechanism of action (Toubi, et al. 2005. Ann NY Acad Sci 1051:506-514, which is incorporated by reference herein in its entirety). In the situation of transplantation, patients with higher levels of Treg cells are more resistant to graft versus host as opposed to patients with lower levels (Zorn, et al. 2005. Blood 106:2903-2911, which is incorporated by reference herein in its entirety). Similarly, a drop in Treg activity is correlated with multiple sclerosis exacerbation/relapse (Haas, et al. 2005. Eur J Immunol 35:3343-3352; Huan, et al. 2005. J Neurosci Res 81:45-52; Viglietta, et al. 2004. J Exp Med 199:971-979, each of which is incorporated by reference herein in its entirety), while treatment with copolymer-1 or TCR-vaccine induced clinical remission and elevated Treg number/activity (Hong, et al. 2005. Proc Natl Acad Sci USA 102:6449-6454; Vandenbark, A. A. 2005. Curr Drug Targets Inflamm Allergy 4:217-229, each of which is incorporated by reference herein in its entirety). Interestingly, it has been proposed that elevated Treg numbers during pregnancy may contribute to the pregnancy-associated remission of multiple sclerosis (Sanchez-Ramon, et al. 2005. Immunol Lett 96:195-201, which is incorporated by reference herein in its entirety).

In the tumor bearing mice Treg cells play an important role in protecting the tumor from immune attack. This is established from experiments demonstrating antigen-specific generation of Tregs, which block tumor immunity (Fu, et al. 2000. Int J Cancer 87:680-687, which is incorporated by reference herein in its entirety), as well as that depletion of Treg allows for unmasking of immunological tolerance and induction of effective immunity (Antony, et al. 2005. J Immunol 174:2591-2601; Nicholl, et al. 2004. J Pediatr Surg 39:941-946; discussion 941-946, each of which is incorporated by reference herein in its entirety). In the clinical situation, enhanced activity and number of Treg cells are associated with poor prognosis in a variety of cancers (Ormandy, et al. 2005. Cancer Res 65:2457-2464; Sasada, et al. 2003. Cancer 98:1089-1099; Unitt, et al. 2005. Hepatology 41:722-730; Jonuleit, et al. 2005. Methods Mol Med 109:285-296, each of which is incorporated by reference herein in its entirety). In addition to the previously mentioned example of ONTAK therapy enhancing antitumor immunity through Treg depletion, other methods of inhibiting Treg are known in the art and can be practiced in the context of the invention disclosed. One method is administration of a dose of cyclophosphamide sufficient to cause decrease in Treg numbers and activity. Such a dose can be between 1-100 mg/m² administered daily orally. Additional variations of this are based on amount of Treg inhibition sought, as determined by ex vivo assays of activity. Yet another method of inhibiting Treg activity is through rendering conventional T cells resistant to Treg mediated suppression. Such resistance can be rendered through activating dendritic cells using TLR-agonists, specifically, TLR4 and 9 agonists (Pasare, et al. 2003. Science 299:1033-1036, which is incorporated by reference herein in its entirety). Accordingly, the invention teaches the use of such agonists such as detoxified LPS, extracellular matrix fragments, heat shock proteins, and CpG DNA for immune stimulation either prior or subsequent to induction of tumor cell death for immune stimulation. Alternatively, another method of overcoming Treg suppression is through administration of TLR-8 agonists, which have been demonstrated to inhibit tumor-derived immune suppression (Peng, et al. 2005. Science 309:1380-1384, which is incorporated by reference herein in its entirety). Administration of said TLR agonists can be performed locally or systemically. One example of systemic administration is through utilizing the commercially available topical formulation of the TLR-7 agonist imiquimod Aldara. This cream may be administered on a daily basis in to a cancer patient on the forearm skin over an area of 5×5 centimeters. While Aldara has been used in the context of dendritic cell maturation for improvement of anti-cancer vaccine effects (Shackleton, et al. 2004. Cancer Immun 4:9; Nair, et al. 2003. J Immunol 171:6275-6282, each of which is incorporated by reference herein in its entirety), the use of Aldara in the context of amplifying responses to endogenous cancer tissue necrosis has not been reported or envisioned in the prior art.

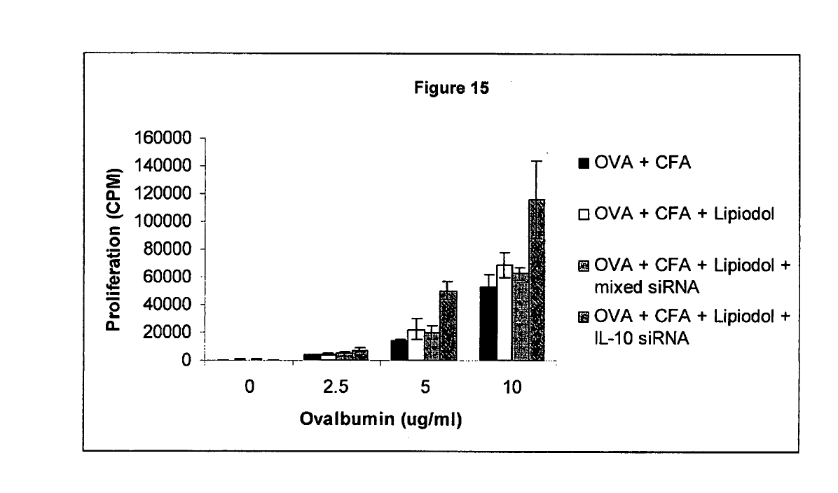

An embodiment of the disclosed invention teaches methods of altering the tumor microenvironment through administration of short interfering RNA specific for immune suppressive factors. We have previously demonstrated that treatment of DC with siRNA is immune modulatory in the tolerogenic sense (Li, et al. 2004. Immunol Res 30:215-230, which is incorporated by reference herein in its entirety). In the series of experiments presented in the Examples section, we know demonstrate that siRNA can be delivered directly into cells in vivo in the form of a vaccine composition comprised of siRNA, lipiodol and CFA. Specifically, FIGS. 15-18 demonstrate that silencing of the IL-10 gene using the lipiodol+CFA method can lead to upregulated recall proliferation, IFN-γ secretion and downregulation of IL-4. These data open an almost endless number of therapeutic possibilities for localized immune modulation through administration of siRNA-lipiodol into tumors. It is to be noted that one skilled in the art understands that both lipiodol and CFA can be substituted for other agents possessing the properties of transfection and localization.

One specific embodiment of the invention is administration of siRNA specific to an immune suppressive factor directly into tumors using a catheter-based delivery approach. Co-administration of the siRNA-lipiodol mixture with embolization, and/or chemotherapy is envisioned within the scope of the invention. A specific application of the invention is generation of siRNA targeting the immune suppressive enzyme indoleamine 2,3-dioxygenase (IDO) (Mellor, A. 2005. Biochem Biophys Res Commun 338:20-24, which is incorporated by reference herein in its entirety), and administering said siRNA via hepatic artery embolization into a patient with liver cancer. Targeting of IDO mRNA transcript is particularly advantageous since in addition to endogenous tumor expression of IDO, host cells upregulate expression of this enzyme in response to immune activation as a negative feedback loop (Kwidzinski, et al. 2005. Faseb J 19:1347-1349, which is incorporated by reference herein in its entirety). Accordingly the silencing of IDO in a cancer patient concurrently with systemic or local immune stimulation can be utilized for synergistic immune enhancement. Numerous other cytokines, transcription factors, and membrane-bound immune suppressive factors can be silenced within the context of the disclosed invention in order to augment immune activation subsequent to induction of localized cell death. Examples of relevant immune suppressive factors associated with neoplasia include: IL-10 (Yang, et al. 2003. Cancer Res 63:2150-2157, which is incorporated by reference herein in its entirety), TGF-β (Chen, et al. 2003. Cytokine Growth Factor Rev 14:85-89, which is incorporated by reference herein in its entirety), Fas ligand (Ryan, et al. 2005. Cancer Res 65:9817-9823, which is incorporated by reference herein in its entirety), VEGF (Ohm, et al. 2003. Blood 101:4878-4886, which is incorporated by reference herein in its entirety), IL-18 binding protein (Paulukat, et al. 2001. J Immunol 167:7038-7043, which is incorporated by reference herein in its entirety), MUC-1 (Chan, et al. 1999. Int J Cancer 82:721-726, which is incorporated by reference herein in its entirety), decoy receptor 3 (Hsu, et al. 2005. J Immunol 175:5135-5145, which is incorporated by reference herein in its entirety), sigma(1) receptors (Zhu, et al. 2003. J Immunol 170:3585-3591, which is incorporated by reference herein in its entirety), heavy chain ferritin (Gray, et al. 2003. Clin Cancer Res 9:2551-2559, which is incorporated by reference herein in its entirety), angiotensin II type I receptor (Smith, et al. 2004. J Inflamm (Lond) 1:3, which is incorporated by reference herein in its entirety), STAT6 (Ostrand-Rosenberg, et al. 2004. Breast Dis 20:127-135, which is incorporated by reference herein in its entirety), or protectin/CD59 (Xu, et al. 2005. Prostate 62:224-232, which is incorporated by reference herein in its entirety).

siRNA may be created using a variety of chemical synthesis methods known to one skilled in the art. Such methods can include addition of phosphorothioate internucleotide linkages, 2′-O-methyl ribonucleotides, 2′-deoxy-2′-fluoro ribonucleotides, “universal base” nucleotides, 5-C-methyl nucleotides, and inverted deoxyabasic residue incorporation. Chemical modifications of the siRNA constructs can also be used to improve the stability of the interaction with the target RNA sequence and to improve nuclease resistance.

In one embodiment, the invention features a chemically modified short interfering siRNA wherein the chemical modification comprises a conjugate covalently attached to the siRNA molecule. In another embodiment, the conjugate is covalently attached to the siRNA molecule via a linker, said linker being degradable within the host or host cells. The conjugate molecule is attached at the 3′-end of either the sense strand, antisense strand, or both strands of the siRNA. The conjugate molecule is attached at the 5′-end of either the sense strand, antisense strand, or both strands of the siRNA. Alternatively the conjugate molecule is attached both the 3′-end and 5′-end of either the sense strand, antisense strand, or both strands of the siRNA, or any combination thereof. In one embodiment, a conjugate molecule of the invention comprises a molecule that facilitates delivery of a siRNA molecule into the tumor cell or host cell surrounding the tumor. In another embodiment, the conjugate molecule attached to the siRNA is a poly ethylene glycol, human serum albumin, or a ligand for a cellular receptor found either on the cancer cell or the proximal host cell that can mediate cellular uptake.

### EXAMPLES

The following examples are given to enable those of ordinary skill in the art to more clearly understand and to practice the present invention. The examples should not be considered as limiting the scope of the invention, but merely as be illustrative and representative thereof.

### Example 1

### Poly (I:C) Administration Increases Proliferative Response to Pvalbumin After Intrahepatic Immunization

Four groups of BALB/c mice (The Jackson Laboratory, Bar Harbor, ME) of 6-8 weeks of age consisted of Group 1 intraperitoneal administration of ovalbumin, Group 2 intrahepatic administration of ovalbumin, Group 3 intrahepatic administration of ovalbumin together with lipiodol, and Group 4 intrahepatic administration of ovalbumin together with lipiodol and Poly (I:C).

Mice in Group 1(5 mice per group) where administered one hundred micrograms of ovalbumin (grade V; Sigma Aldrich) dissolved in 0.1 mL of 0.9% saline solution intraperitoneally. The following procedures were performed for mice receiving intrahepatic immunization: Mice were anesthetized with an intraperitoneal injection of ketamin at a concentration of 0.075 mg/g and medetomidine 0.005 mg/g (Sigma Aldrich, St Louis, Mo.). A midline abdominal incision was made, and the viscera were exposed. One hundred micrograms of ovalbumin was dissolved in 0.1 mL of 0.9% saline solution was injected into the portal vein with a 30-gauge needle for mice in Group 2. Mice in Group 3 received one hundred micrograms of ovalbumin dissolved in 0.1 mL of lipiodol (Guerbet, Roissy CdG Cedex, France). Mice in Group 4 received one hundred micrograms of ovalbumin dissolved in 0.1 mL of lipiodol with 5 micrograms of Poly (IC) stabilized in carboxymethylcellulose (Sigma). After the injection, the needle was rapidly withdrawn, and hemostasis was secured without hematoma formation by gentle pressure with 2-mm3 gelfoam (Advance Biofactures, Lynbrook, N.Y.). Mice undergoing complications during the procedure were replaced with additional mice. Seven days after the primary immunization, all mice were boosted by administration of 100 micrograms of ovalbumin in a 50 microliter solution of complete Freund's adjuvant (CFA; Difco Laboratories, Detroit, Mich.). Fourteen days after the secondary immunization, the mice were killed and splenic lymphocytes were isolated for the assessment of cellular proliferation recall response. Cells were cultured at a concentration of 1×10⁵ cells/well in 96 cell plates, in 200 μl of RPMI 1640 (Life Technologies) supplemented with 10% FCS (Life Technologies), 100 U/ml of penicillin (Life Technologies), and 100 μg/ml of streptomycin (Life Technologies). Cells were cultured at 37° C. in a humidified atmosphere of 5% CO₂ for 3 days in the presence of the indicated about of ovalbumin, and pulsed with 1 μCi of [³H]thymidine (Amersham Pharmacia Biotech) for the last 16 h of culture. Cells were harvested onto glass fiber filters, and the radioactivity incorporated was quantitated using a Wallac Betaplate liquid scintillation counter. Results were expressed as the mean cpm of triplicate cultures±SEM.

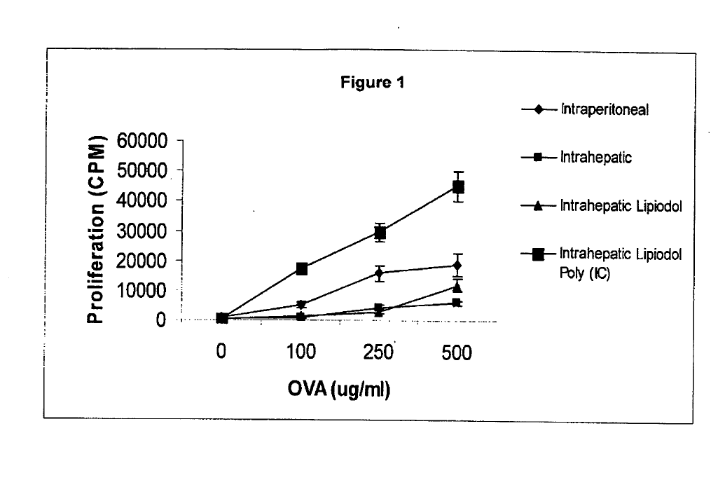

As illustrated in FIG. 1, a profound reduction in proliferative response was observed in Group 2 and 3 mice, which received intravenous hepatic immunization in comparison to Group 1 mice which were immunized intraperitoneally. The intraperitoneal route was chosen as a control since the immunogenicity of subcutenous immunization is established to be much more potent than hepatic immunization. Numerous publications in the art use the intraperitoneal route as a control for hepatic immunization. While the suppression of recall response was not effected by the presence of lipiodol, the addition of Poly (I:C) resulted in a profound stimulation of recall response as seen Group 4 mice.

### Example 2

### Poly (I:C) Administration Increases Interferon Gamma Response to Ovalbumin After Intrahepatic Immunization

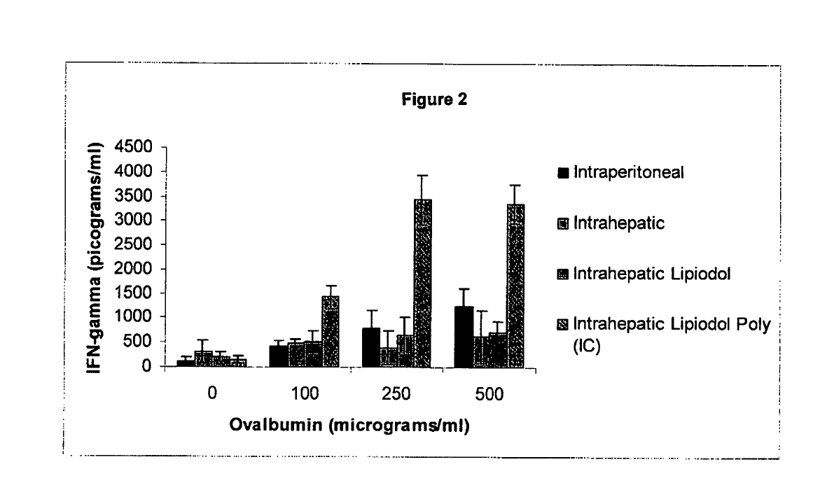

The experimental conditions of the above example were duplicated with the purpose of identifying whether the heightened proliferative response observed in the Group 4 treated mice could also be seen at the level of cytokine production. Indeed the association between interferon gamma production and cytolytic/cytoinhibitory function of T cells is established in the art. In order to detect cytokine production supernatants were harvested from the tissue culture plates at 48 hours of stimulation with ovalbumin and analyzed by interferon gamma ELISA (Quantikine murine IFN-γ ELISA; R&D Systems, Minneapolis, Minn.). As illustrated in FIG. 2, a profound upregulation of interferon gamma secretion was observed in T cells responding to ovalbumin in vitro. This indicates that the ability of the lipiodol-Poly (IC) mixture to potentiate immune responses is not restricted to proliferative recall response but also to secretion of Type I cytokines such as interferon gamma.

### Example 3

### Poly (I:C) Administration Increases DTH Response to Ovalbumin After Intrahepatic Immunization

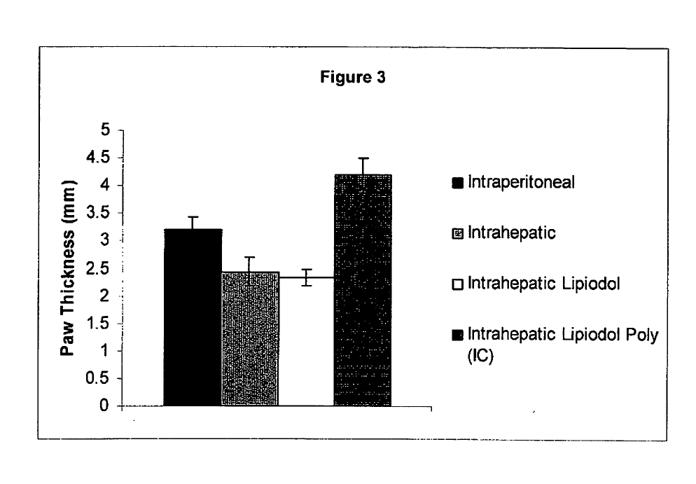

The experimental conditions of the above example were duplicated with the purpose of identifying whether the heightened proliferative response observed in the Group 4 treated mice could also be seen at the level of delayed type hypersensitivity response. Measurement of the footpad thickness (with skinfold calipers) was performed 24 hours after the subcutaneous footpad injection of ovalbumin (50 μg of heat-aggregated ovalbumin in 10 μL of saline. The footpad injection was performed 14 days after the intraperitoneal boosting described in Example 1. As observed in FIG. 3, a significant increase in footpad swelling was observed in the mice having received the lipiodol-Poly (IC) intrahepatic immunization. This indicates that the potentiation of immunity was not limited to proliferative and cytokine responses, but also to functional inflammation.

### Example 4

### Poly (I:C) Administration Abrogates Need for Boosting Induce Proliferative Response

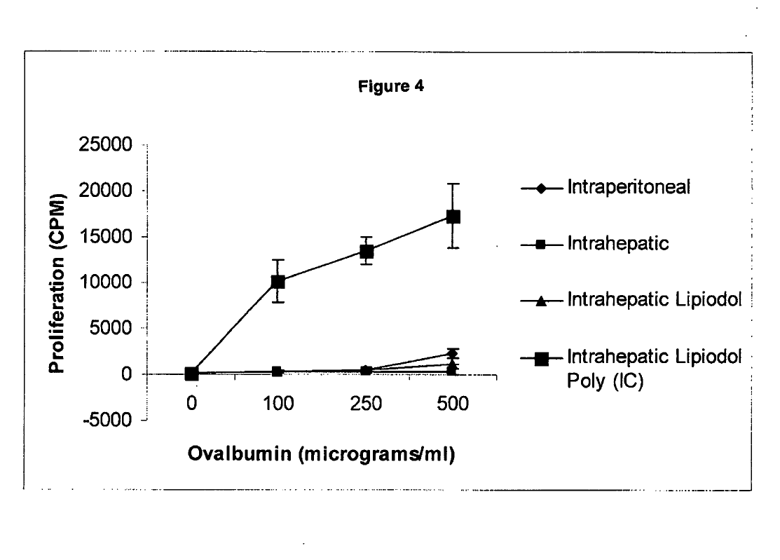

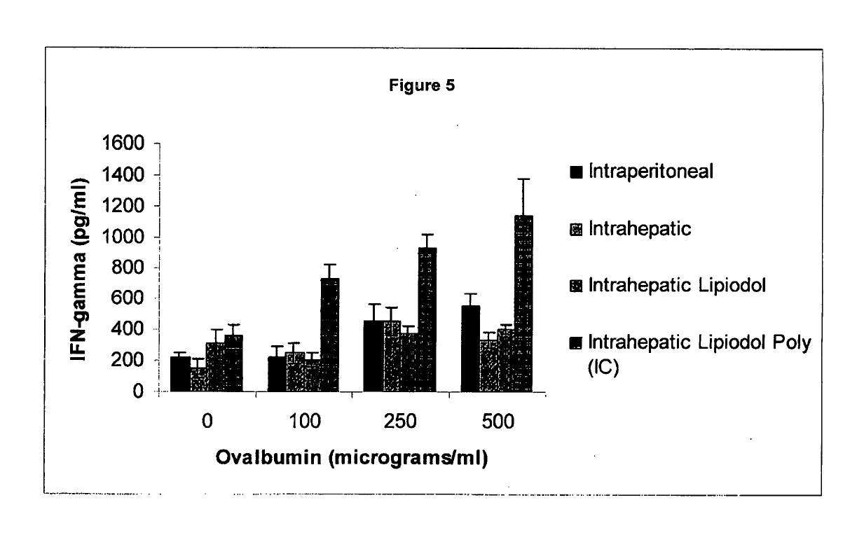

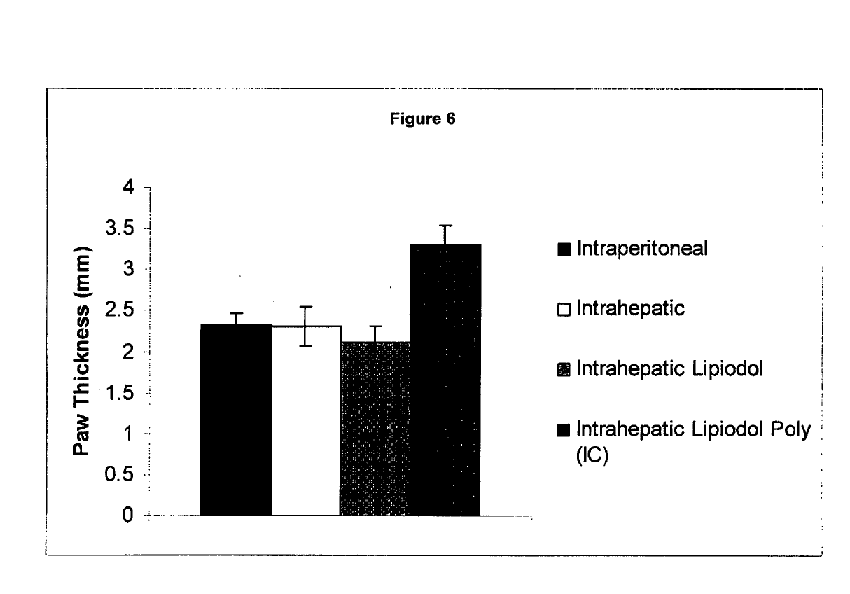

The experimental system of Example 1 was repeated with the absence of the boosting step at day-7 post intra-hepatic ovalbumin administration. As observed in FIG. 4, the proliferative response of T cells from mice having received the lipiodol-Poly (IC) intrahepatic immunization was substantially greater than the Group 1-3 control mice. This was also observed at the level of interferon gamma production (FIG. 5) and DTH response (FIG. 6).

### Example 5

### In vitro and in vivo Immune Modulation by Lipiodol-siRNA Mixtures

Animals:

Female C57/BL6 and BALB/c mice (The Jackson Laboratories, Bar Harbor, Me.), 5 wk of age, were kept in filter-top cages at the Animal Care and Veterinary Services Facility, the University of Western Ontario according to the Canadian Council for Animal Care Guidelines. Mice were fed by food and water ad libitum and allowed to settle for 2 wk before initiation of experimentations.

DC Generation and siRNA Transfection:

At Day 0, bone marrow cells were flushed from the femurs and tibias of C57/BL6 mice, washed and cultured in 6-well plates (Corning, N.Y.) at 4×10⁶ cells/well in 4 ml of complete medium (RPMI 1640 supplemented with 2 mM L-glutamine, 100 U/ml penicillin, 100 μg of streptomycin, 50 μM 2-ME, and 10% FCS (all from Life Technologies, Ontario, Canada) supplemented with recombinant GM-CSF (10 ng/ml; PeproTech, Rocky Hill, N.J.) and recombinant mouse IL-4 (10 ng/ml; PeproTech). All cultures were incubated at 37° C. in 5% humidified CO₂. Non-adherent cells were removed after 48 h of culture (Day 2) and fresh medium was added. After 7 days of culture, >90% of the cells expressed the characteristic DC-specific marker CD11c as determined by FACS. DC were washed and plated in 24-well plates at a concentration of 2×10⁵ cells/well in 400 μl of serum-free RPMI 1640. Transfection with GenePorter, lipiodol, or naked siRNA was performed as described below on day 7 of culture.

siRNA Synthesis and Transfection:

siRNA sequences were selected according to the method previously used by us (Hill, et al. 2003. J Immunol 171:691-696, which is incorporated by reference herein in its entirety). siRNA specific for IL-12p35 (AACCUGCUGAAGACCACAGAU) (SEQ ID NO: 1), IL-10 (AATAAGCTCCAAGAGAAAGGC) (SEQ ID NO: 2) or mismatched (mixed) control sequence (AACTGCCAGATGGATGGTGAC) (SEQ ID NO: 3) were synthesized and annealed by the manufacturer (Dharmacon, Lafayette, Colo.). In some experiments siRNA was admixed with lipiodol and CFA. In others it was and added at a concentration of 60 pMol to DC cultures. For transfection, 3 μl of 20 μM annealed siRNA were incubated with 3 μl of GenePorter (Gene Therapy Systems, San Diego, Calif.) or lipiodol (Ultra-Fluide™ Laboratoire Guerbet, France) in a volume of 100 μl of RPMI 1640 (serum free) at room temperature for 30 min. This was then added to 400 μl of DC cell culture as described above. Mock controls were transfected with 3 μl of GenePorter alone. For naked siRNA, addition of the same concentration of siRNA was performed and procedures were repeated in an identical manner with the exception of addition of transfection reagent. After 4 h of incubation, an equal volume of RPMI 1640 supplemented with 20% FCS was added to the cells. Twenty-four hours later, transfected DC were washed and used for subsequent experiments. DC activation was performed in 24-well plates by stimulation with LPS (10 ng/ml; Sigma-Aldrich, St. Louis, Mo.) plus TNF-(10 ng/ml; PeproTech) for 24 hours.

Flow Cytometry:

Phenotypic analysis of DC was performed using flow cytometry on a FACScan (Becton Dickninson, San Jose, Calif.) and analyzed using CellQuest software (BD Biosciences). The cells were stained with FITC-conjugated mAb against surface markers associated with DC maturation: anti-mouse CD11c, anti-mouse CD40, anti-mouse CD80, and anti-mouse CD86 (Cedarlane Laboratories, Mississauga, ON). Ig of the same isotype were used as controls. Annexin V and propidium iodine analysis for apoptosis, necrosis was performed using the Apotag kit (Cedarlane Laboratories, Hornby Ontario, Canada).

Mixed Lymphocyte Reaction:

C57/BL6 DC after transfection were irradiated (3,000 rad) and seeded in triplicate at various concentrations in a flat-bottom 96-well plate (Coming) for use as stimulator cells. Splenic T cells from BALB/c mice were isolated by gradient centrifugation over Ficoll-Paque (Amersham Pharmacia Biotech, Quebec) and T cell nylon wool column purification, and added as responders (5×10⁵ cells/well). The mixed lymphocytes were cultured at 37° C. for 72 h in 200 μl of RPMI 1640 supplemented with 10% FCS, 100 U/ml of penicillin, and 100 μg/ml of streptomycin and pulsed with 1 μCi/well of ³H-labelled thymidine (Amersham Pharmacia Biotech) for the last 16 h of culture. Cells were harvested onto glass fiber filters, and the radioactivity incorporated was quatitated using a Wallac Betaplate liquid scintillation counter (Beckman, Fullerton, Calif.). Results were expressed as the mean counts per min of triplicate cultures±SEM. In some experiments anti-IL-10 (JES5 2A5, Pharmingen) or isotype control antibody were added for the duration of the MLR at a concentration of 5 ug/ml.

Immunization with Ag/Lipiodol Mixture:

C57/BL6 mice were immunized intradermally at the interior side of both hind legs with 100 μl of KLH or ovalbumin (1 μg/μl) emulsified in CFA (Difco Laboratories, Detroit, Mich.) in the presence or absence of 10 nMol siRNA and 10% lipiodol. After 14 days mice were sacrificed and T cells extracted as described above.

Proliferation Assays:

Proliferative recall responses to KLH and ovalbumin in immunized mice were assessed by sacrificing C57/BL6 mice 14 days after immunization with antigen-loaded DC. T cells were purified from suspensions of lymph nodes using CD4+ T cell column (R&D Systems) after washing in PBS. Purified T cells were cultured in 96 well plates with irradiated syngeneic splenocytes in triplicate and mixed with serial dilutions of KLH or OVA at concentrations ranging from 0-10 ug/ml. Following a 72-h incubation, 1 μCi of [³H]thymidine (Amersham) was added to each well for 16 h. Using an automated cell harvester, the cells were collected onto glass microfiber filter, and the radioactive labeling incorporation was measured by a Wallac Betaplate liquid scintillation counter.

ELISA:

The supernatants from recall response T cell cultures or MLR were harvested and assessed for DC cytokines (IL-12p70, IL-10) and T cell cytokines (IFN-, IL-4) by ELISA. Cytokine-specific ELISA (Endogen, Rockford, Ill.) was used for detecting cytokine concentrations in culture supernatants according to the manufacturer's instructions using a Benchmark Microplate Reader (Bio-Rad, Hercules, Calif.).

### Example 6

### Lipiodol Enhances Transfection of Functional siRNA into DC

We have previously demonstrated that transfection of DC with siRNA specific for the p35 component of IL-12 induces potent gene specific silencing at the mRNA transcript level as demonstrated by RT-PCR and subsequently reduced expression of the IL-12 p70 heterodimer as witnessed by ELISA protein (Hill, et al. 2003. J Immunol 171:691-696, which is incorporated by reference herein in its entirety). Furthermore, we and others have reported that siRNA can be endocytosed into dendritic cells (DC) and other cell types in absence of transfection reagent both in vitro and in vivo (Li, et al. 2004. Immunol Res 30:215-230; Ichim, et al. 2004. Am J Transplant 4:1227-1236; Klein, et al. 2003. Gastroenterology 125:9-1, each of which is incorporated by reference herein in its entirety). Accordingly, we chose to investigate whether lipiodol can be used to increase uptake of siRNA in DC in using an in vitro system.

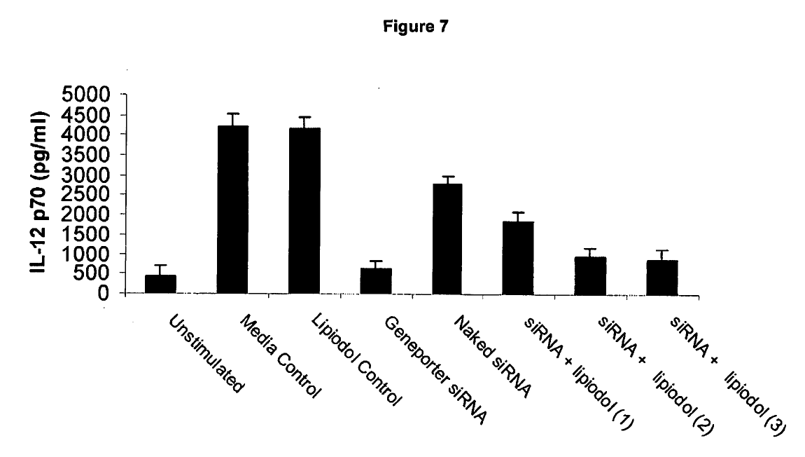

In our laboratory, the combination of 10 ng/ml of LPS and TNF-α, respectively, (LPS/TNF) is used as a standard method of inducing activation of bone marrow derived DC for production of IL-12, as well as upregulation of costimulatory molecules such as CD40, CD80 and CD86 (Ichim, et al. 2003. Transpl Immunol 11:295-306, which is incorporated by reference herein in its entirety). Using this stimulation system, and assessing IL-12 p70 production by ELISA, we sought to determine the potency of mixed lipiodol with siRNA to p35 at suppressing IL-12 production. Naked siRNA, and siRNA in various concentrations of lipiodol was administered to day-7 bone marrow derived C57/BL6 DC. Activation by LPS/TNF was performed on day 8 while culture supernatants were assessed for IL-12 production on day 10 of culture. It was observed that siRNA transfection with GenePorter induced a potent (>90%) inhibition of IL-12 production and that the naked siRNA induced a smaller (>30%) inhibition. Transfecting the siRNA with lipiodiol at concentration of 2 and 3 μl/well induced a significantly stronger inhibition of IL-12 production (>75%) as compared to naked siRNA, but not to the same extent as GenePorter (FIG. 7). Similarly to our previously published experiments, administration of mismatched siRNA had no inhibitory effect on production of IL-12 (data not shown). Furthermore, although it has previously been reported that lipiodol is not cytotoxic even at high concentrations (Bhattacharya, et al. 1996. Br J Cancer 73:877-881, which is incorporated by reference herein in its entirety), we wanted to discount the possibility that lipiodol was mediated non-specific killing of DC. Viability assays using annexin-V and PI staining and analyzed by flow cytometry demonstrated no increase in apoptosis or necrosis in comparison to untreated DC (data not shown). Overall, these data support the notion that lipiodol is an easy to use method of transfecting bone marrow derived DC in vitro DC.

### Example 7

### Immune Modulation by Lipiodol/siRNA: Upregulation of IL-10

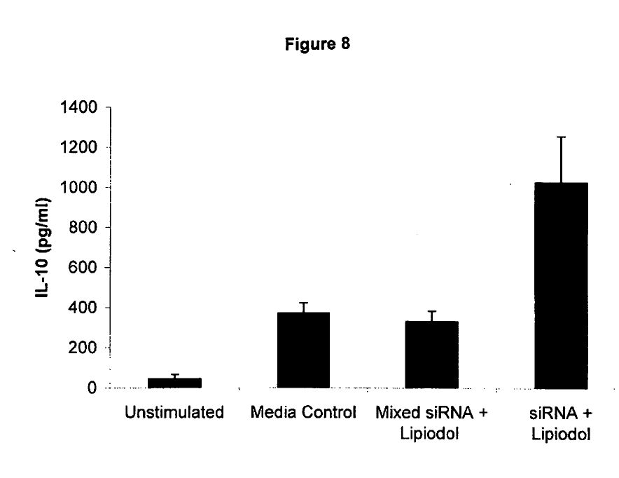

We have previously demonstrated that DC silenced for the IL-12p35 subunit possess an increased production of IL-10 and are poor stimulators of MLR (Hill, et al. 2003. J Immunol 171:691-696, which is incorporated by reference herein in its entirety). Using the 3 ul concentration of lipiodol found most effective at inhibiting IL-12 production in FIG. 7, it was determined whether the lipiodol/siRNA treated DC possessed the same immunomodulatory properties as previously reported by us. Indeed, we observed that siRNA transfected by lipiodol induced a specific increase in IL-10 production by LPS/TNF stimulated DC as seen in FIG. 8.

### Example 8

### Immune Modulation by Lipiodol/siRNA: Inhibition of Allogeneic T Cell Proliferation

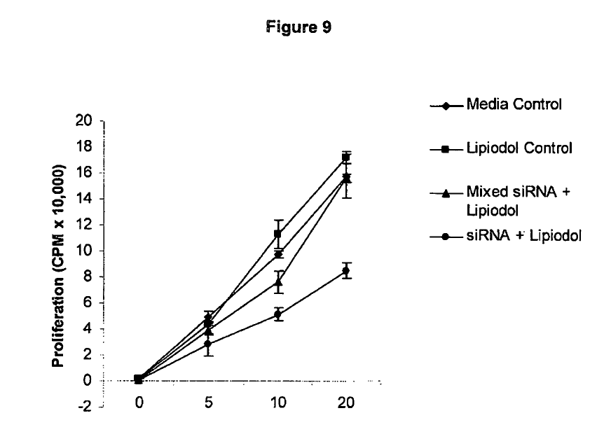

Since the specific function of DC in vivo is stimulation of T cell responses, we sought to determine whether the siRNA/lipiodol mixture had effects on inhibition of mixed lymphocyte reaction (MLR). Indeed, the siRNA specific for IL-12 p35 and not the mismatched (mixed) control inhibited proliferation of responding T cells in a 3 day MLR with naive BALB/c splenocytes (FIG. 9). Furthermore, as seen in FIG. 9, the lipiodol alone did not suppress allostimulatory ability of the DC, indicating that the siRNA itself was specifically inducing inhibition.

### Example 9

### Immune Modulation by Lipiodol/siRNA: Inhibition of Allogeneic T Cell Interferon Gamma Production

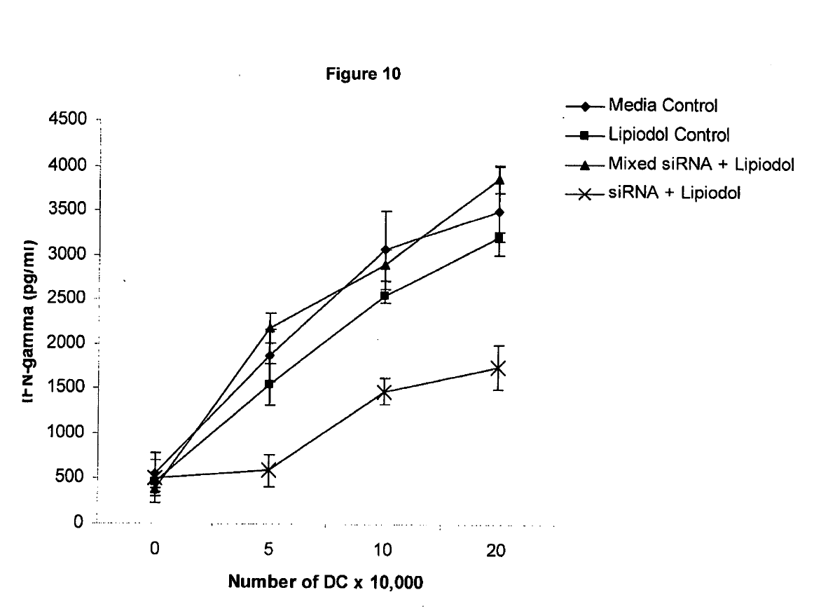

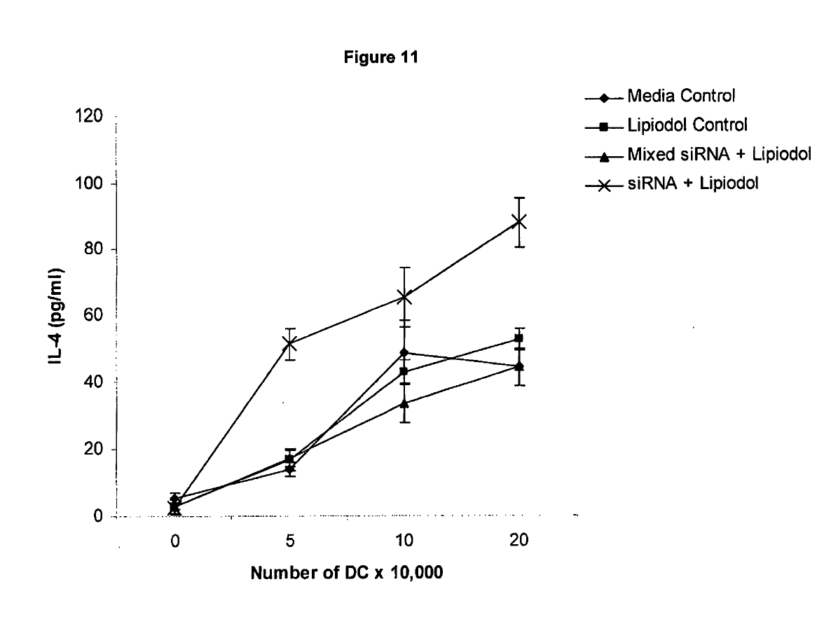

When supernatants of the MLR were assayed at 48 hours for the prototypic Th1 cytokine IFN-γ an profound inhibition of production of this cytokine was observed (FIG. 10), indicating that the immunomodulatory effects of lipiodol-siRNA can specifically alter not only proliferation but also cytokine release of allogeneic responding T cells. Accordingly, the levels of the prototypic Th2 cytokine, IL-4 were increased in T cells responding to the lipiodol-siRNA treated DC (FIG. 11).

### Example 10

### Lipiodol/siRNA Induces Th1>Th2 Shift in vivo

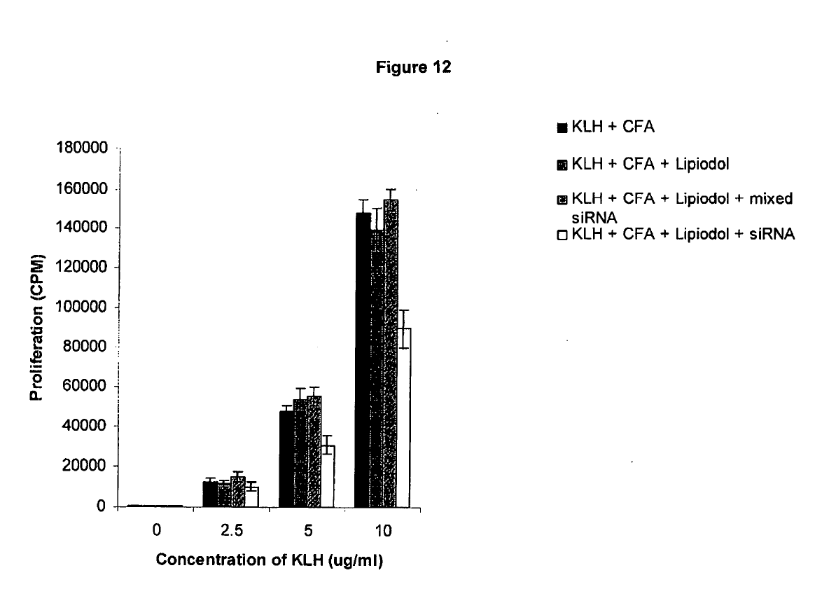

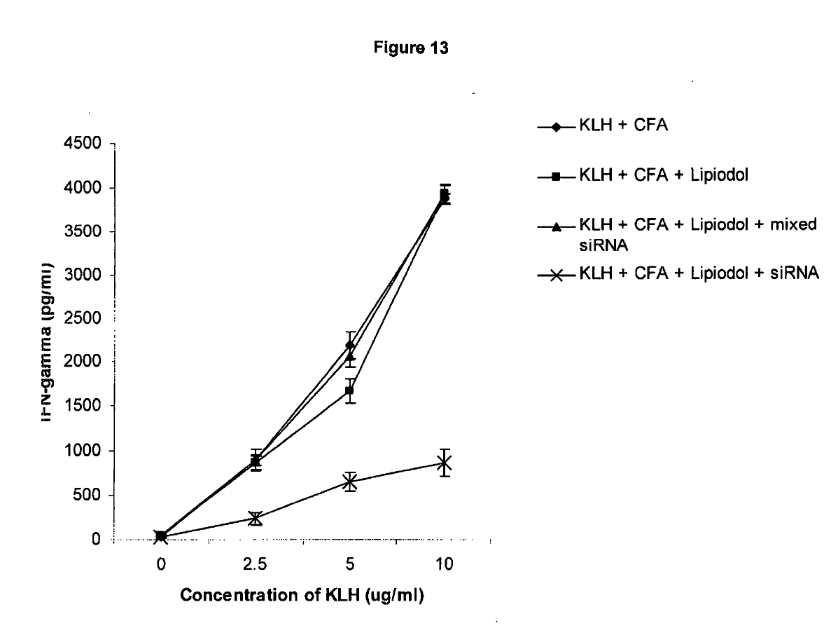

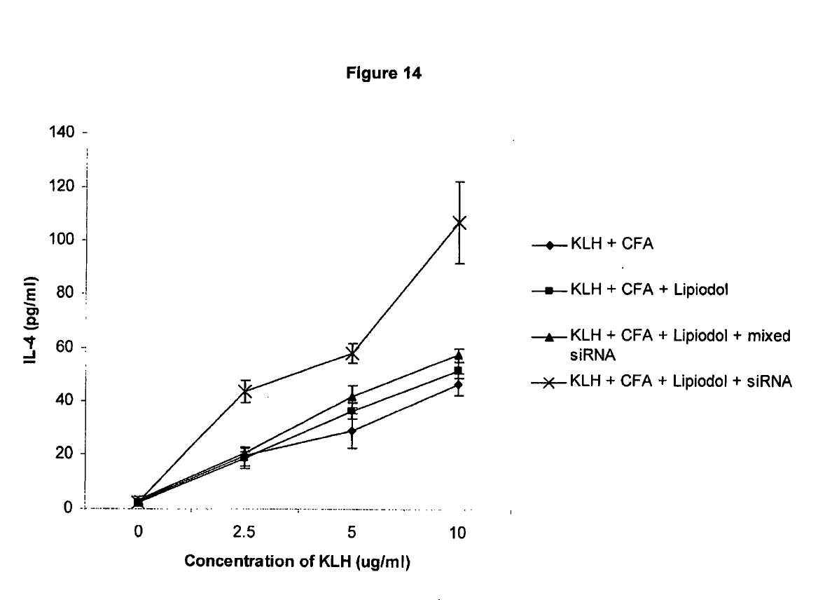

Having demonstrated above that lipiodol can serve as a transfection reagent for uptake of siRNA in DC, combined with the fact that lipiodol is commonly used for a variety of clinical applications (Vogl, et al. 2005. Radiology 234:917-922; Di Stefano, et al. 2005. Radiology 234:625-630, each of which is incorporated by reference herein in its entirety), we assessed whether lipiodol/siRNA can be used to modulate immune responses in vivo. Accordingly, we used the KLH recall response as an indicator. C57BL/6 mice were immunized with the IL-12 siRNA/lipiodol mixture combined with CFA and KLH. 14 days following immunization, recall response experiments were performed using isolated CD4 T cells from draining lymph nodes as previously described by us (Hill, et al. 2003. J Immunol 171:691-696, which is incorporated by reference herein in its entirety). Proliferative responses for KLH was significantly inhibited in the lipiodol/siRNA treated animals at restimulation concentrations of 5 and 10 ug/ml (FIG. 12). When supernatants were harvested and analyzed by ELISA for cytokine response, a Th1>Th2 shift was observed as demonstrated by higher production of IFN-γ (FIG. 13) and IL-4 (FIG. 14), respectively. In order to assess for antigen specificity, in some experiments, mice were immunized with siRNA/lipiodol with KLH and concurrently injected with mixed siRNA/lipiodol and OVA. The recall response to KLH was suppressed in terms of proliferation and possessed a Th2 cytokine profile, whereas the response to OVA was not immune modulated (data not shown). Overall, these experiments demonstrate that siRNA can be administered both in vitro and in vivo for immune modulatory purposes using lipiodol as a carrier.

### Example 11

### Immune Stimulation by Lipiodol-siRNA Targeting IL-10

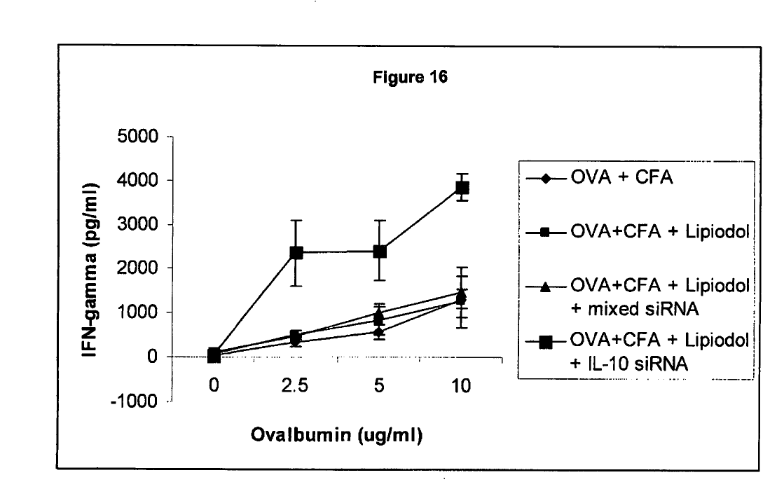

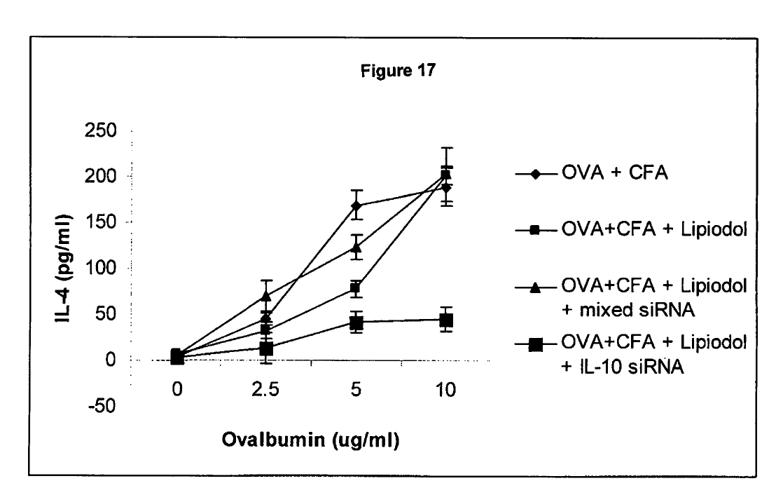

Having demonstrated immune suppressive activities of siRNA targeting IL-12p35 when delivered in vivo in a lipiodol/CFA composition, we next assessed whether substitution of siRNA targeting IL-10 would induce the reverse effects on recall proliferative and cytokine responses. Using the identical protocol of the experiment above, we observed an increased recall proliferative response to KLH (FIG. 15), an enhanced IFN-γ secretion (FIG. 16), and a reduced production of IL-4 (FIG. 17). Although these results were anticipated, this experiment demonstrates conclusively that the administration of siRNA using lipiodol can be a method of immune modulation both from an inhibitory, and stimulatory perspective.

What is claimed is:

1. A method of inducing an anticancer immune response in a cancer patient in need thereof through the steps of:
a. Admixing a concentration of immune stimulant with a clinically applicable localizing agent and a single or plurality of agents capable of causing localized cell death;
b. Administering said combination directly into the tumor and/or arteries providing the tumor with blood supply; and
c. Administering an embolizing agent in the proximity of the tumor and/or directly into the arteries providing the tumor with blood supply.

2. The method of claim 1 wherein the immune stimulant is a small molecule, a nucleic acid, a protein, or a combination thereof.

3. The method of claim 2 wherein said small molecule immune stimulant is selected from a group comprising of: muramyl dipeptide, thymosin, 7,8-disubstituted guanosine, imiquimod, detoxified lipopolysaccharide, isatoribine and alpha-galactosylceramide.

4. The method of claim 2 wherein said nucleic acid is selected from a group comprising of: short interfering RNA targeting the mRNA of immune suppressive proteins, CpG oligonucleotides, Poly IC, unmethylated oligonucleotides, plasmid encoding immune stimulatory molecules, or chromatin-purified DNA.

5. The method of claim 2 wherein said protein is selected from one of the following compounds: IL-2, IL-7, IL-8, IL-12, IL-15, IL-18, IL-21, IL-23, IFN-α, β, γ, TRANCE, TAG-7, CEL-1000, bacterial cell wall complexes, or LIGHT.

6. The method of claim 1 wherein said agent capable of causing cell death is a chemotherapeutic or radiotherapeutic agent.

7. The method of claim 1 wherein the localizing agent is an iodinated oil mixture

8. The method of claim 7 wherein the localizing agent is lipiodol.

9. The method of claim 1 wherein the embolizing agent is selected from a group comprising of: Avitene, Gelfoam, Occlusin and Angiostat.

10. A method of inducing an anticancer immune response in a patient in need thereof through the steps of:
a. Admixing a concentration of short interfering RNA with a clinically applicable localizing agent and a single or plurality of agents capable of causing localized cell death;
b. Administering said combination directly into the tumor and/or arteries providing the tumor with blood supply; and
c. Administering an embolizing agent in the proximity of the tumor and/or directly into the arteries providing the tumor with blood supply.

11. The method of claim 10 wherein the short interfering RNA is administered in the one of the following forms: DNA plasmids capable of transcribing hairpin loop RNA which is subsequently cleaved by endogenous cellular processes into short interfering RNA, double stranded RNA chemically synthesized oligonucleotides, in vitro generated siRNA fragments from mRNA.

12. The method of claim 11 wherein the short interfering RNA is targeted to one or more mRNA selected from the following group: IDO, IL-4, IL-10, TGF-β, FGF, and VEGF.

13. The method of claim 10 wherein cell death is caused by a chemotherapeutic or radiotherapeutic agent, or by embolization of the tumor.

14. A pharmaceutical composition capable of eliciting an antigen-specific immune response to tumor derived proteins comprising of:
a. An immune stimulant
b. A clinically applicable localizing agent and;
c. An agent capable of causing cell death
d. An embolizing agent

15. The composition of claim 14 wherein the immune stimulant is a small molecule, a nucleic acid, a protein, or a combination thereof.

16. The composition of claim 14 wherein said small molecule immune stimulant is selected from one of the following compounds: muramyl dipeptide, thymosin, 7,8-disubstituted guanosine, imiquimod, detoxified lipopolysaccharide, isatoribine or alpha-galactosylceramide.

17. The composition of claim 15 wherein said nucleic acid is selected from a group comprising of: short interfering RNA targeting the mRNA of immune suppressive proteins, CpG oligonucleotides, Poly IC, unmethylated oligonucleotides, plasmid encoding immune stimulatory molecules, and chromatin-purified DNA.

18. The composition of claim 15 wherein said protein is selected from one of the following compounds: IL-2, IL-7, IL-8, IL-12, IL-15, IL-18, IL-21, IL-23, IFN-α, β, γ, TRANCE, TAG-7, CEL-1000, bacterial cell wall complexes, or LIGHT.

19. The composition of claim 14 wherein said agent capable of causing cell death is a chemotherapeutic or radiotherapeutic agent.

20. The composition of claim 14 wherein cell death is caused by embolization of the tumor with an embolizing agent that is selected from a group comprising of: Avitene, Gelfoam and Angiostat,

21. A method of modification of the transcatheter chemoembolization procedure in order to induce an antitumor immune response to in a patient with hepatic cancer in need thereof consisting of the steps of:
a. Selecting a patient suitable for therapy
b. Inserting a catheter into said patient
c. Administering a mixture of a single or plurality of immune stimulant(s) admixed with a clinically applicable localizing agent and/or with a single or plurality of agents capable of causing localized cell death;
d. Administering said combination directly into the tumor and/or arteries providing the tumor with blood supply using said catheter;
e. Administering an embolizing agent in the proximity of the tumor and/or directly into the arteries providing the tumor with blood supply using said catheter;
f. Assessing the levels of immune activation;
g. Providing subsequent agents to enhance/maintain immune activation;
h. Performing the procedures of steps “b” to “e” as needed determined by the level of immune activation and/or tumor regression.

22. The method of claim 21 wherein said patient meets the current standard of care inclusion/exclusion criteria for eligibility for transcatheter chemoembolization.

23. The method of claim 22 wherein said patient suffers from a localized primary hepatocellular carcinoma, or a hepatically-located metastasis originating from a tumor exterior to the liver.

24. The method of claim 23 wherein said tumor is a functional neuroendocrine cancer such as a carcinoid tumor or a pancreatic endocrine tumor.

25. The method of claim 24 wherein said cancer patient failed systemic therapy with octreotide to control carcinoid syndrome.

26. The method of claim 23 wherein said tumor is unresectable, or tumor growth control is desired until a liver transplant is feasible.

27. The method of claim 26 wherein said patient has adequate hepatic function as determined by a plasma concentration of bilirubin <2 mg/dl; plasma albumin of >2.7g/dl; and no portal vein occlusion.

28. The method of claim 27 wherein said patient has adequate renal function as determined by plasma concentration of creatinine <2mg/dl.

29. The method of claim 21 wherein said catheter is inserted using the Seldinger technique, and passed under fluoroscopic control into the hepatic artery determined to be the tumor feeding artery.

30. The method of claim 21 wherein the mixture injected into the tumor feeding artery comprises a composition of Poly (IC), lipiodol, and doxorubicin at a concentration sufficient to induce localized tumor cell death, immune activation, and form a localized depot.

31. The method of claim 21 wherein the mixture injected into the tumor feeding artery comprises a composition of an immune stimulant, lipiodol, and a chemotherapeutic agent at a concentration sufficient to induce localized tumor cell death, immune activation, and form a localized depot.

32. The method of claim 21 wherein the immune stimulant is capable of activating expression of immune stimulatory molecules on cells of the localized microenvironment.

33. The method of claim 21 wherein the chemotherapeutic agent is capable of activating expression of immune stimulatory molecules on cells of the localized microenvironment.

34. The method of claim 33 wherein the chemotherapeutic agent is melphalan/

35. The method of claim 21 wherein the chemotherapeutic agent is capable of upregulating antigenic expression of tumor cells.

36. The method of claim 35 wherein said chemotherapeutic agent is selected from a group comprising of 5-azacytidine, sodium phenylbutyrate, and trinchostatin A.

37. The method of claim 21 wherein said immune stimulant is a protein, or a combination thereof.

38. The composition of claim 37 wherein said small molecule immune stimulant is selected from one of the following compounds: muramyl dipeptide, thymosin, 7,8-disubstituted guanosine, imiquimod, detoxified lipopolysaccharide, isatoribine or alpha-galactosylceramide.

39. The composition of claim 37 wherein said nucleic acid is selected from a group comprising of one of the following: short interfering RNA targeting the mRNA of immune suppressive proteins, CpG oligonucleotides, Poly IC, unmethylated oligonucleotides, plasmid encoding immune stimulatory molecules, or chromatin-purified DNA.

40. The composition of claim 37 wherein said protein is selected from a group comprising of: IL-2, IL-7, IL-8, IL-12, IL-15, IL-18, IL-21, IL-23, IFN-α, β, γ, TRANCE, TAG-7, CEL-1000, bacterial cell wall complexes, or LIGHT.

41. The method of claim 39 wherein the short interfering RNA is administered in the one of the following forms: DNA plasmids capable of transcribing hairpin loop RNA which is subsequently cleaved by endogenous cellular processes into short interfering RNA, double stranded RNA chemically synthesized oligonucleotides, in vitro generated siRNA fragments from mRNA.

42. The method of claim 39 wherein the short interfering RNA is targeted to one or more mRNA selected from a group comprising of: IDO, IL-4, IL-10, TGF-β, FGF, VEGF.

43. The method of claim 21 wherein the embolizing agent is selected from a group comprising of: Avitene, Gelfoam, Occlusin and Angiostat.

44. The method of claim 21 wherein said cell death causing agent is a radiotherapeutic.

45. The method of claim 21 wherein said cell death is caused or accelerated from a group of therapeutic approaches comprising of: radiofrequency ablation, localized hyperthermia, conformal radiotherapy, and antibody-target radiotherapeutics.

46. The method of claim 21 wherein immune activation state is assessed in an antigen-specific or non-antigen-specific manner.

47. The method of claim 21 wherein said antigen-specific immune activation is quantitated by the numbers of tetramer positive T cells identified by staining with a tetramer bearing a tumor antigen.

48. The method of claim 47 wherein said antigen is specific for liver carcinoma

49. The method of claim 47 wherein said antigen is selected from a group comprising of MAGE peptides, NY-ESO-1b peptide, and alpha-fetoprotein derived peptides.

50. The method of claims 47-49 wherein said T cells are tetramer positive and express interferon gamma spontaneously or upon ex vivo restimulation.

51. The method of claim 50 wherein said T cells are examined for expression of function and cleaved T Cell Receptor zeta-chain.

52. The method of claim 51 wherein said T cells are examined for ability to proliferate ex vivo in response to antigen challenge.

53. The method of claim 46 wherein said antigen specific immune response is assessed by ability of the patient immune response to form a delayed type hypersensitivity reaction to antigenic sources selected from group comprising of: autologous tumor cell lysates, allogeneic tumor cell lysates, MAGE peptides, NY-ESO-1b peptide, and alpha-fetoprotein derived peptides.

54. The method of claim 46 wherein a model antigen such as ovalbumin or keyhole limpet hemocyanin is originally administered as part of the chemoembolization mixture and immune response to it is assessed by methods selected from a group comprising of: tetramer positivity for said antigen, expression of functional TCR zeta chain on tetramer positive cells for said antigen, proliferative response to said antigen ex vivo, cytokine production ability in response to said antigen ex vivo, and ability to generated delayed type hypersensitivity reactions to said antigen.

55. The methods of claims 46 to 54 wherein T cell memory formation in response to the described antigens and antigenic compositions is assessed by expression of markers associated with either T cell central memory or T cell effector memory phenotypes.

56. The method of claim 55 wherein T cell central memory cells are positive for expression of CD45RO, CCR7 whereas T cell effector memory cells are positive for expression of CD45RO and negative for expression of CCR7.

57. The method of claim 46 wherein immune response is assessed through assaying non-antigen specific measurements of immune activation selected from a group comprising of: T cell proliferative, cytokine, and activation marker responses to ex vivo stimuli such as conconavalin A, phyohemmaglutinin, anti-CD3 together with anti-CD28.

58. The method of claim 46 wherein non-antigen specific immune activation is assessed by the cytokine release and natural cytotoxicity of natural killer, and/or natural killer T cells.

59. The method of claim 46 wherein antibody mediated responses are assessed as a measure of antigen-specific immune stimulation.

60. The method of claim 21 wherein a dose of an agent capable of maintaining/augmenting an immune response is administered at a timepoint and concentration sufficient to increase immunological control of the neoplasia.

61. The method of claim 21 wherein the agent capable of increasing immune response is administered prior to the embolization procedure.

62. The method of claim 61 wherein the agent is a T cell depleting agent administered in such as manner so as to cause a state of homeostatic lyphoproliferative expansion before the embolization procedure.

63. The method of claim 61 wherein such agent being selected from a group comprising of: radiotherapy, cyclophosphamide, Campath, and anti-CD3.

64. The method of claim 61 wherein as immunization with tumor antigen is performed subsequent to the embolization procedure.

65. The method of claim 61 wherein a subsequent embolization procedure is the method of tumor immunization.

66. The method of claim 61 wherein agents capable of inducing memory cell turnover are administered for immune stimulation.

67. The method of claim 61 wherein said agents are selected from a group comprising of IFN-alpha, IL-12, IL-15, IL-18, and IL-23.

68. The method of claim 61 an agent capable of inducing expression of cytokines selected from a group comprising of IFN-alpha, IL-12, IL-15, IL-18, and IL-23 are administered.

69. The method of claim 61 wherein said agents are agonists of toll like receptors.

70. The method of claim 61 wherein one said agent is imiquimod.

71. A method of altering the hepatic microenvironment as to make it inhospitable for tumor growth comprising the steps of:
a. Introducing into said hepatic microenvironment an agent capable of immune stimulation;
b. Concurrently adding a localizing agent; and
c. Adjusting said dose based on immunological parameters known in the art to prevent engraftment of metastatic tumors.

72. A method of preconditioning the liver microenvironment prior to induction of localized tumor cell death, so as to enhance the ability of the immune response to induce anti-tumor effectors subsequent to induction of tumor cell death.

73. The method of claim 72 wherein preconditioning is achieved through activation of hepatic natural killer t cells.

74. The method of claim 72 wherein the activation of natural killer T cells is accomplished through administration of an agent that indirectly induces activation of said natural killer T cells through stimulating production of activitory compounds by hepatic dendritic cells.

75. The method of claim 72 wherein said agent is selected from a group comprised of one or more of the following: Poly IC, muramyl dipeptide, thymosin, 7,8-disubstituted guanosine, imiquimod, detoxified lipopolysaccharide, isatoribine or alpha-galactosylceramide.

76. The method of claim 72 wherein prior to administration of a dendritic cell activator, said dendritic cell numbers are enhanced through supplying an effective amount of DC progenitor proliferative stimuli.

77. The method of claim 72 wherein said DC progenitor proliferative stimuli is selected from a group comprising of fms-like tyrosine kinase-3 ligand, GM-CSF, progenipoietin-1, and thrompoietin.

78. A method of immune modulating the systemic host prior to induction of tumor cell death in order to enhance the ability of the immune response to induce anti-tumor effectors subsequent to induction of localized tumor cell death.

79. The method of claim 78 wherein systemic repair of T cell abnormalities is accomplished through administration of a sufficient dose of anti-oxidants selected from a group comprising of: n-acetylcysteine, ascorbic acid, genistein, co-enzyme Q-10, alpha lipoic acid, and vitamin E.

80. The method of claim 78 wherein an agent capable of reducing the activation threshold necessary for T cell activation is added.

81. The method of claim 80 wherein said agent is selected from a group comprising of: antagonistic anti-CTLA-4 antibodies, agonisting anti-CD28 antibodies, depleting anti-CD25 antibodies, low dose IL-2, and a TLR agonist.

82. A method of systemically immune modulating the host subsequent to induction of localized tumor cell death so as to enhance the ability of the immune response to induce anti-tumor effectors subsequent to induction of localized tumor cell death.

83. The method of claim 82 wherein systemic repair of T cell abnormalities is accomplished through administration of a sufficient dose of anti-oxidants selected from a group comprising of: n-acetylcysteine, ascorbic acid, genistein, co-enzyme Q-10, alpha lipoic acid, and vitamin E.

84. The method of claim 82 wherein an agent capable of reducing the activation threshold necessary for T cell activation is added.

85. The method of claim 84 wherein said agent is selected from a group comprising of: antagonistic anti-CTLA-4 antibodies, agonisting anti-CD28 antibodies, depleting anti-CD25 antibodies, low dose IL-2, and a TLR agonist.

86. A method of effecting immune modulation in a host in need thereof through administration of short interfering RNA in a composition of lipiodol.

87. The method of claim 86 wherein siRNA is administered in the form of a therapeutic vaccine in combination with an adjuvant.

88. The method of claim 87 wherein said adjuvant is selected from a group comprising of: QS-21, complete Freund's adjuvant, incomplete Freund's adjuvant, agonistic anti-CD40 antibody, Montanide ISA-51, and IL-12.

89. The method of claim 88 wherein said adjuvant is a TLR agonist.

90. The method of claim 89 wherein said TLR agonist is imiquimod.

91. The method of claim 86 wherein said siRNA hybridizes with the transcript of an immune suppressive molecule.

92. The method of claim 89 wherein said immune suppressive molecule is selected from a group comprising of: IL-10, TGF-β, Fas ligand, VEGF, IL-18 binding protein, decoy receptor 3, heavy chain ferritin and protectin/CD59 (183).

93. The method of claim 92 wherein said siRNA is administered at a concentration sufficient to induce the process of RNA interference.

94. The method of claim 93 wherein said siRNA is administered in a composition of lipiodol, with procedures and compositions known to induce necrosis of tumor cells.

95. The method of claim 94 wherein said procedures are selected from a group comprising of: transcatheter chemoembolization, transcatheter embolization, radiofrequency ablation, localized hyperthermia, conformal radiotherapy, and antibody-target radiotherapeutics.
